(19) United States
(12) Patent Application Publication
(10) Pub. No.: US 2025/0025650 A1
(43) Pub. Date: Jan. 23, 2025

(54) BREATHING ASSISTANCE APPARATUSES AND/OR COMPONENTS THEREOF AND/OR USES THEREOF

(71) Applicant: Fisher & Paykel Healthcare Limited, Auckland (NZ)

(72) Inventors: David Martin Russell, Auckland (NZ); Samuel Carey Mathew Sanson, Auckland (NZ); Russel William Burgess, Auckland (NZ); Yi Lin Huang, Auckland (NZ)

(21) Appl. No.: 18/558,271

(22) PCT Filed: May 5, 2022
(86) PCT No.: PCT/IB2022/054141

§ 371 Date: Oct. 31, 2023

Related U.S. Application Data
(60) Provisional application No. 63/184,695, filed on May 5, 2021.
(60) Provisional application No. 63/362,188, filed on Mar. 30, 2022.

(51) Int. Cl. A61M 16/00; A61M 16/10

(52) U.S. Cl. CPC A61M 16/022; A61M 16/0003; A61M 16/1095; A61M 2205/3561

(57) ABSTRACT

Disclosed is an apparatus configured to operate in at least one therapy mode and at least one non-therapy mode. After a predetermined time operating in the at least one non-therapy mode, the apparatus is configured to: transmit the data to a device, and/or receive a software package from a or the device, and/or receive therapy parameters from a or the device, and/or update parameters of the apparatus.

### TECHNICAL FIELD

The present disclosure relates to various breathing assistance apparatuses and/or components thereof and/or uses thereof.

### BACKGROUND ART

Breathing assistance apparatuses are used to deliver a flow of gas to patients in various environments (such as hospital, medical facility, residential care, or home environments). A breathing assistance apparatus (e.g., a flow therapy apparatus and/or a pressure therapy apparatus) may include an oxygen inlet that enables the respiratory apparatus to deliver supplemental oxygen with the flow of gas. A breathing assistance apparatus may also (or alternatively) include an ambient air inlet and a flow generator (for example including a blower) to provide gases to a patient. A breathing assistance apparatus may also (or alternatively) include a humidification apparatus that enables the respiratory apparatus to deliver heated and humidified gases. A breathing assistance apparatus may allow adjustment of, and control over, characteristics of the gases flow. These characteristics may include for example flow rate, temperature, gas concentration (such as supplemental oxygen concentration), humidity, and pressure, etc.

Patients suffering from various health conditions and diseases can benefit from breathing assistance (for example respiratory therapy). In at least one form, the respiratory therapy may be oxygen therapy. For example, a patient suffering from chronic obstructive pulmonary disease (COPD), pneumonia, asthma, bronchopulmonary dysplasia, heart failure, cystic fibrosis, sleep apnea, lung disease, trauma to the respiratory system, acute respiratory distress, and/or other conditions or diseases can benefit from respiratory therapy. Similarly, patients receiving pre- and post-operative oxygen delivery can also benefit from respiratory therapy. By way of further example patients suffering from obstructive sleep apnea (OSA) may also benefit from respiratory therapy (for example CPAP and/or Bi-level therapies)

### SUMMARY

In a first aspect of the disclosure there is provided a breathing assistance apparatus comprising:
a flow generator configured to generate a flow of gases,
a humidifier configured to be pneumatically connected to the flow generator and to humidify the flow of gases,
wherein the apparatus is configured to be connected to a conduit that conveys the flow of gases
wherein the apparatus is configured to operate in at least one therapy mode and at least one non-therapy mode, wherein while operating in the at least one therapy mode the apparatus is configured to provide therapy to a user,
wherein the apparatus is configured to collect and store data, the data comprising therapy data collected during operation in the at least one therapy mode and/or apparatus data,
wherein after a predetermined time operating in the at least one non-therapy mode, the apparatus is configured to:
a) transmit the data to a device, or
b) receive a software package from a or the device, or
c) receive therapy parameters from a or the device, or
d) update parameters of the apparatus, or
e) any combination of a)-e).

When the apparatus is operating in the at least one therapy mode, therapy may be provided to the user.

The at least one therapy mode may comprise:
a) a Continuous Positive Airway Pressure (CPAP) mode,
b) a Bubble Continuous Positive Airway Pressure (BCPAP) mode,
c) a Nasal High Flow (NHF) mode,
d) a Bilevel mode
e) any combination of a)-d).

The apparatus may comprise a controller that is configured to operate the apparatus according to any of the therapy modes a)-d) listed above.

The apparatus (and optionally a controller of the apparatus) may be configured to control a flow generator and/or the humidifier in accordance with at least one therapy mode.

Each therapy mode may have one or more associated therapy parameters

Wherein the associated therapy parameters comprise one or more of: a therapy flow rate of the gases, a therapy pressure support level, a therapy temperature of the gases, therapy humidity of the gases, a therapy temperature at the end of the breathing conduit.

Each therapy mode may have associated software configured to be executed by the apparatus to control the apparatus to provide the specific therapy.

When the apparatus is operating in the at least one non-therapy mode, no therapy may be being provided to the user.

The apparatus may be configured to automatically operate in the at least one non-therapy mode after completion of the at least one therapy mode.

The apparatus may be configured to enter the at least one non-therapy mode after receiving an end of therapy command (optionally generated via an input from a user interface, and/or by detection that the patient interface has been removed from the user).

The end of therapy command may be generated:
a) via an input from a user interface,
b) by detection that the patient interface has been removed from the user
c) by detection that the patient interface has been removed from the user for a predetermined amount of time
d) any combination of a)-c).

When the apparatus is operating in the at least one non-therapy mode, the flow generator may be activated and is generating the flow of gases.

A user may also select a therapy mode via the user interface. The controller can receive a user input i.e. a user selection of a therapy mode via the user interface and operate the apparatus accordingly to provide a therapy as per the selected therapy mode.

When the apparatus is operating in the at least one non-therapy mode, the flow generator may be providing the flow of gases at:
a flow rate less than a therapy flow rate of the flow of gases provided during a therapy mode and/or,
a predetermined flow rate and/or,
a predetermined motor speed.

In some configurations given the predetermined flow rate may be provided by controlling the predetermined motor speed. This may be by, for example, a look up table or formula defining the relationship between flow rate and motor speed. In non-therapy modes this approach may allow for easier control of the flow generator as no patient will be connected, and the resistance to flow may be known (i.e., in disinfection mode), or considered constant (i.e., in drying mode and/or disinfection mode).

When the apparatus is operating in the at least one non-therapy mode the humidifier (optionally a heater plate of the humidifier) may be activated and optionally the humidifier may be configured to humidify the flow of gases.

When the apparatus is operating in the at least one non-therapy mode a heater of the conduit may be activated and may be configured to heat the flow of gases in the conduit.

The at least one non-therapy mode may comprise a drying mode configured to dry the conduit.

When the apparatus is operating in the drying mode a heater of the conduit may be controlled while the flow generator provides gases at a predetermined flow rate.

When the apparatus is operating in the drying mode a heater of the humidifier may be controlled to a predetermined value (optionally the predetermined value is a predetermined power, and the predetermined power may be less than about 5% or less than about 10% of a maximum power provided to the heater plate), or the heater plate may be deactivated during the drying mode.

The heater of the conduit may be controlled to a predetermined temperature at the end of the conduit or controlled to a predetermined duty cycle, or to a predetermined voltage, or to a predetermined current or to a predetermined power.

The predetermined duty cycle may be 100%.

The predetermined temperature may be greater than 45 degrees Celsius.

The drying mode may be configured to be operated for about 20 minutes to about 120 minutes, or about 90 minutes.

The drying mode may comprise controlling the flow generator to provide a predetermined flow generator output, wherein the flow generator output may be a motor speed about 1000 RPM to about 3000 RPM or less than about 2000 RPM.

The drying mode may comprise controlling the flow generator to provide a predetermined flow rate, wherein the predetermined flow rate may be about 5 litres/minute to about 20 litres/minute.

The drying mode may be configured to evaporate remaining condensate in the apparatus and/or patient breathing conduit and/or patient interface.

The non-therapy mode may be a warm up mode.

The apparatus may operate in the warm up mode when the apparatus is powered on.

The warm up mode may comprise controlling a heater of the conduit to control a temperature at the end of the conduit to a desired temperature.

The desired temperature at the end of the conduit may be based on one or more therapy parameters of the apparatus.

The one or more therapy parameters may be:
a) a therapy chamber outlet temperature,
b) a therapy dew point temperature (at the chamber outlet or at the end of the conduit),
c) a therapy humidity (at the chamber outlet or at the end of the conduit) and/or
d) a therapy temperature at the end of the conduit
e) any combination of a)-e).

The desired temperature at the end of the conduit may be a predetermined temperature.

The end of the conduit temperature may be within about 2 degrees Celsius to about 5 degrees Celsius, or about 2.5 degrees Celsius of the desired patient end temperature, and optionally, about 2 degrees Celsius to about 5 degrees Celsius, or about 2.5 degrees Celsius less than the predetermined temperature or the therapy parameter.

The warm up mode may comprise controlling a heater of the humidifier to a predetermined temperature, to a predetermined duty cycle, or to a predetermined voltage, or to a predetermined current, or to a predetermined power.

The warm up mode may comprise deactivating the flow generator.

The conduit may be configured to convey the gases to a patient via a patient interface.

The non-therapy mode may be a disinfection mode.

In the disinfection mode, the apparatus may be configured to be connected to a disinfection conduit.

In the disinfection mode a heater of the disinfection conduit may be controlled so that the flow of gases in the disinfection conduit reach a predetermined temperature.

The predetermined temperature may be about 50 degrees Celsius to about 100 degrees Celsius, or about 60 degrees Celsius to about 90 degrees Celsius.

The disinfection conduit may comprise a temperature sensor.

The disinfection mode may comprise controlling the flow generator to provide a predetermined flow rate, wherein the predetermined flow rate is about 10 litres/minute to about 20 litres/minute.

The non-therapy mode may be a standby mode.

The standby mode may comprise running the flow generator at a predetermined flow rate, or a predetermined motor speed.

The predetermined flow rate may be lower than a therapy flow rate being provided to the patient.

The predetermined motor speed may be about 1000 RPM to about 3000 RPM or less than about 2000 RPM.

The therapy data may comprise data relating to the user and/or therapy provided to the user.

The therapy data may comprise:
a) an oxygen saturation (SpO2) of the user,
b) a respiratory rate of the user,
c) a humidity (dew point) of the gases provided to the user,
d) a flow rate of the gases provided to the user,
e) a tidal volume of the user,
f) a minute ventilation of the user
g) any combination of a)-f).

The therapy data may comprise:
a) usage data of the apparatus,
b) answers to one of more questions provided to a user,
c) a therapy report (optionally the therapy report relates to previous therapy session report and/or a current finished therapy session),
d) the results of a health enquiry,
e) any combination of a)-d).

The apparatus may comprise one or more sensors configured to determine the therapy data.

When the apparatus is operating in the at least one non-therapy mode the user is presented with one or more questions, and provides answers to these questions via at least one user interface, and wherein the questions and/or answers to the questions form part of the therapy data.

The apparatus may be configured receive a sensor output from the one or more sensors, wherein the therapy data may be based on the sensor output from one or more sensors.

The one or more sensors may be located within a housing of the apparatus.

The one or more sensors may be located remotely to the apparatus.

The therapy data may be from the at least one therapy mode.

The therapy data may comprise data from at least historic therapy mode.

The therapy data may comprise data from at least one therapy mode which has not previously been transmitted to the device.

The apparatus data may relate to one or more properties of the apparatus and/or the surrounding environment of the apparatus.

The data may be transmitted to the device even if the apparatus has not operated in at least one therapy mode.

The data may be transmitted to the device after a predetermined period of time has elapsed since a last transmission of data to the device (optionally the predetermined period of time is 24 hours).

The device may be:
a) a server
b) a local device
c) a remote device
d) any combination of a)-c)

The predetermined time may be about 5 minutes to about 25 minutes.

The predetermined time may be greater than 5 minutes.

The predetermined time may be less than a total length of time the apparatus is configured to operate in the non-therapy mode.

The predetermined time may be a proportion of a total length of time the apparatus is configured to operate in the non-therapy mode.

The apparatus may comprise a controller configured to control operation of the apparatus.

The user may be presented with a visual indicator and/or an audio indicator if the user attempts to power off the apparatus when the data is being transmitted to the device.

The user may be presented with a visual indicator and/or an audio indicator while the data is being uploaded to the device.

To ensure that the apparatus is not powered off immediately after the apparatus exits the therapy mode, the apparatus may be configured to:
a) transmit the data to a device, or
b) receive a software package from a or the device, or
c) receive therapy parameters from a or the device, or
d) update parameters of the apparatus, or
e) any combination of a)-e).

after the predetermined time operating in the at least one non-therapy mode.

After a predetermined time operating in the at least one non-therapy mode, the apparatus may be configured to activate a network interface and establish a connection with the device to transmit data to the device, and/or receive a software package and/or therapy parameters from a or the device.

The software package may comprise one or more of:
a) a firmware update,
b) a software update.

After receiving the software package, the apparatus may apply the software package to the apparatus.

Updating a parameter of the apparatus may comprise undertaking a sensor recalibration.

After receiving the therapy parameters from a or the device, the apparatus may apply updates the therapy parameters of the apparatus.

The apparatus may only receive the software package and/or the therapy parameters if they are an update to a current software package, and/or a current therapy parameter.

The apparatus may have a gases inlet and gases outlet, and the conduit may be configured to connect to the gases outlet.

The apparatus may comprise one or more sensors (optionally a temperature sensor) located in the flow of gases.

The apparatus may comprise one or more sensors (optionally a temperature sensor) located in the gases outlet.

The apparatus may comprise a housing, and the flow generator and/or humidifier are located in the housing.

After a predetermined time operating in the non-therapy mode the apparatus is configured to: update parameters of the apparatus and then transmit the data to a device.

After a predetermined time operating in the non-therapy mode the apparatus is configured to: transmit the data to a device, and then receive a software package from a or the device and then update parameters of the apparatus.

After a predetermined time operating in the non-therapy mode the apparatus is configured to: update parameters of the apparatus, and then transmit the data to a device, and then receive a software package from a or the device, and then update parameters of the apparatus based on the software package received from a or the device.

The apparatus may be configured to receive therapy parameters from a or the device after transmitting the data to a device

The apparatus may be configured to receive therapy parameters from a or the device after receiving the software package from a or the device.

The apparatus may be configured to receive therapy parameters from a or the device before updating parameters of the apparatus.

The apparatus may comprise at least one display (optionally as part of a display module).

When operating in a non therapy mode, the apparatus may display information relating to the non therapy on the display.

The information may be one or more of: the type of non therapy mode, an indication that the non therapy mode is active, a time remaining in the non therapy mode, a warning not to use the apparatus.

The updated parameters of the apparatus may be sensor calibration parameters (and optionally sensor calibration parameters) of one or more sensors.

The sensor calibration parameters may relate to a relationship between an output of the one or more sensors and the characteristic the sensor is configured to measure.

The one or more sensor may comprises:
a flow rate sensor configured to measure a flow rate of the flow of gases, optionally the flow rate sensor comprises an ultrasonic sensor, and/or
an oxygen concentration sensor configured to measure an oxygen concentration of the gases, optionally the oxygen concentration sensor comprises an ultrasonic sensor.

When no supplemental gas is provided as part of the flow of gases, the apparatus may be configured to determine an output of the oxygen concentration sensor indicative of an oxygen concentration of the flow of gases, wherein based on the output of the oxygen concentration sensor and an estimated oxygen ambient concentration the apparatus is configured to determine an oxygen concentration sensor calibration parameter.

The estimated oxygen ambient concentration may be about 19% to about 23% about 20.9%, or about 21%, or about 22%.

When supplemental gases are provided as the flow of gases, the apparatus may be configured to determine an output of the oxygen concentration sensor indicative of an oxygen concentration of the flow of gases, wherein based on the output of the oxygen concentration sensor and a predetermined oxygen concentration the apparatus is configured to determine an oxygen concentration sensor calibration parameter.

In another aspect of the disclosure, there is provided a breathing assistance apparatus comprising:
a gases inlet and a gases outlet,
a housing,
a flow generator located within the housing, the flow generator configured to generate a flow of gases,
a humidifier located within the housing in fluid communication with the flow generator and configured to humidify the flow of gases from the flow generator, the humidifier comprising a heater configured to heat a fluid in a humidification chamber of the humidifier,
a conduit configured to be connected to the gases outlet and convey the flow of gases, the conduit comprising a heater configured to heat the flow of gases in the conduit,
one or more sensors located within the housing,
a controller comprising at least a processor and memory, the controller configured to control at least the flow generator, the humidifier and the heater of the conduit, wherein the controller is configured receive a sensor output from the one or more sensors and to store data based on the sensor output from one or more sensors,
wherein the controller is configured to operate the apparatus in at least a therapy mode and a non-therapy mode, wherein in the therapy mode the apparatus is configured to provide therapy to a user according to one or more therapy parameters, and in the non-therapy mode at least one of the flow generator, the heater of the humidifier and/or the heater of the conduit are activated, and no flow of gases is provided to the user,
wherein after a predetermined time operating in the non-therapy mode the controller is configured to:
a) transmit the data to a device, or
b) receive a software package from a or the device, or
c) receive therapy parameters from a or the device, or
d) update parameters of the apparatus, or
e) any combination of a)-d)

In another aspect of the disclosure, there is provided a breathing assistance apparatus comprising:
a gases inlet and a gases outlet,
a housing,
a flow generator located within the housing, the flow generator configured to generate a flow of gases,
a humidifier located within the housing in fluid communication with the flow generator and configured to humidify the flow of gases from the flow generator, the humidifier comprising a heater configured to heat a fluid in a humidification chamber of the humidifier,
a conduit configured to be connected to the gases outlet and convey the flow of gases, the conduit comprising a heater configured to heat the flow of gases in the conduit,
one or more sensors located within the housing,
a controller comprising at least a processor and memory, the controller configured to control at least the flow generator, the humidifier and the heater of the conduit, wherein the controller is configured receive a sensor output from the one or more sensors and to store data based on the sensor output from one or more sensors,
wherein the controller is configured to operate the apparatus in at least a therapy mode and a drying mode, wherein in the therapy mode the apparatus is configured to provide therapy to a user according to one or more therapy parameters, and in the drying mode at the heater of the humidifier is deactivated, and a heater of the conduit is activated while the flow generator provides gases at a predetermined flow rate and/or a predetermined motor speed of a motor of the flow generator,
wherein after at least 10 minutes operating in the drying mode the controller is configured to:
a) transmit the data to a device, or
b) receive a software package from a or the device, or
c) receive therapy parameters from a or the device, or
d) update parameters of the apparatus, or
e) any combination of a)-d)

After a predetermined time operating in the at least one non-therapy mode, the apparatus may be configured to activate a network interface and establish a connection with the device.

The apparatus may further comprise one or more remote sensors is located remotely to the apparatus.

The controller may be configured receive a sensor output from the one or more remote sensors, wherein the controller is configured to store data based on the sensor output from the one or more remote sensors.

After receiving the software package from a or the device, the apparatus may be configured to apply the software package.

The apparatus may check the software package version and update the software package on the apparatus if it is an older version.

The apparatus may transmit to the device a confirmation message once the software package is installed successfully and/or transmits an error report if the software package is not installed successfully.

After receiving the therapy parameters from a or the device (as a prescription update), the apparatus may be configured to update the therapy parameters of the apparatus based on the received therapy parameters.

Receiving a software package and receiving therapy parameters may be initiated by a fetch operation by the apparatus.

The apparatus may be configured to query the device as to whether any updated software packages and/or updated therapy parameters are available, and if updated software packages and/or updated therapy parameters are available then the apparatus is configured to receive the software package and/or therapy parameters.

A breathing assistance apparatus comprising:
a gases inlet and a gases outlet,
a housing,
a flow generator located within the housing, the flow generator configured to generate a flow of gases,
a humidifier located within the housing in fluid communication with the flow generator and configured to humidify the flow of gases from the flow generator, the humidifier comprising a heater configured to heat a fluid in a humidification chamber of the humidifier,
a conduit configured to be connected to the gases outlet and convey the flow of gases, the conduit comprising a heater configured to heat the flow of gases in the conduit,
one or more sensors located within the housing,
a controller comprising at least a processor and memory, the controller configured to control at least the flow generator, the humidifier and the heater of the conduit, wherein the controller is configured receive a sensor output from the one or more sensors and to store data based on the sensor output from one or more sensors,
wherein the controller is configured to operate the apparatus in at least a therapy mode and a warm up mode, wherein in the therapy mode the apparatus is configured to provide therapy to a user according to one or more therapy parameters, and in the warm up mode the heater of the humidifier and the heater of the breathing conduit are activated, while the flow generator is deactivated,
wherein after 10 minutes operating in the warm up mode the controller is configured to:
a) transmit the data to a device, or
b) receive a software package from a or the device, or
c) receive therapy parameters from a or the device, or
d) update parameters of the apparatus, or
e) any combination of a)-d)

A breathing assistance apparatus comprising:
a flow generator configured to generate a flow of gases,
a humidifier configured to be pneumatically connected to the flow generator and to humidify the flow of gases,
wherein the apparatus is configured to be connected to a conduit that conveys the flow of gases
wherein the apparatus is configured to operate in at least one therapy mode and at least one non-therapy mode, wherein while operating in the at least one therapy mode the apparatus is configured to provide therapy to a user,
wherein the apparatus is configured to collect and store data, the data comprising therapy data collected during operation in the at least one therapy mode and/or apparatus data,
wherein a device is configured to send a notification to the apparatus that an update in therapy parameters, or a software package is available,
wherein the apparatus is configured to download the updated therapy parameters and/or the updated software package from the device,
wherein after a predetermined time operating in the at least one non-therapy mode, the apparatus is configured to apply the updated therapy parameters and/or the updated software package.

When the apparatus is operating via a battery as a power source the apparatus may, after the predetermined time in a non-therapy mode, not undertake the action, or may prompt the user to confirm the action before undertaking the action.

When the apparatus is operating via a battery as a power source a user may be able to manually prompt the apparatus to undertake the action (optionally after the predetermined time in a non-therapy mode).

When the apparatus is reconnected to an external power source, the apparatus may prompt the user to perform the action (optionally after the predetermined time in a non-therapy mode).

When the apparatus is entered into a charging state, the apparatus may be configured to immediately perform the action or perform the action after a predetermined time in the charging state (optionally after the predetermined time in a non-therapy mode).

The predetermined time may be the predetermined time as described above in relation to operating in a non-therapy mode, or more than about 2 minutes, or more than about 5 minutes.

In some embodiments, the charging state may be entered into when the apparatus is not powered on.

When a service task is completed (for example general servicing of the apparatus, or a replacement of a component of the apparatus) the apparatus may be configured to immediately perform the action or perform the action a predetermined time after the service task is complete.

In some embodiments, after the service task is complete the apparatus may prompt the user (in this case a service technician) to undertake the action (as described above).

In the context of the action being performed after a service task, the action may act as a connectivity test to ensure that the communications module is operational.

When the apparatus is entered into a travel state (for example for air travel or travel away from a user's normal location) the apparatus may, after the predetermined time in a non-therapy mode, not undertake the action, or may prompt the user to confirm the action before undertaking the action.

When the apparatus exits the travel state, the apparatus may prompt the user to perform the action.

When the apparatus is in a travel state a user may be able to manually prompt the apparatus to undertake the action.

In a travel state the apparatus may deactivate the communication module (or part of the communications module).

In a travel state the apparatus may also operate via a battery.

In another aspect of the disclosure, there is provided a breathing assistance apparatus comprising:
a flow generator configured to generate a flow of gases,
a humidifier configured to be pneumatically connected to the flow generator and to humidify the flow of gases,
wherein the apparatus is configured to operate in at least one therapy mode and at least one non-therapy mode, wherein while operating in the at least one therapy mode the apparatus is configured to provide therapy to a user,
wherein when the apparatus is operating in the at least one non-therapy mode, the apparatus is configured to update at least one parameter of the apparatus.

The apparatus may be configured to update parameters of the apparatus after a predetermined time of the apparatus operating in the at least one non-therapy mode.

The apparatus may be configured to update parameters of the apparatus after a non therapy mode has been completed.

The updated parameters of the apparatus may be sensor calibration parameters (and optionally sensor calibration parameters) of at least one sensor.

The apparatus may be configured to update sensor calibration parameters of the at least one sensor at least once, and optionally a plurality of times.

The apparatus may be configured to update sensor calibration parameters of at least one sensor, and after a predetermined time update sensor calibration parameters of the at least one sensor again.

The sensor calibration parameters may relate to a relationship between an output of the at least one sensor and the characteristic the sensor is configured to measure.

The sensor calibration parameters may be stored in a memory of the apparatus.

The sensor calibration parameters may be used by the apparatus to determine the characteristic the sensor is configured to measure based on the output of the at least one sensor.

The sensor calibration parameters may be used by the apparatus in a therapy mode.

The sensor calibration parameters may comprise one or more of:
a calibration factor,
a calibration curve,
an internal parameter of the sensor.

The apparatus may be configured to update a control scheme based on the sensor calibration parameters.

The apparatus may be configured to update the sensor calibration parameters based on the output of the at least one sensor and a further sensor.

The at least one sensor and the further sensor may be configured to measure the same characteristic.

The at least one sensor may comprise a first pressure sensor and the further sensor comprises a second pressure sensor, and the apparatus is configured to update the sensor calibration parameters of a first pressure sensor based on the output of a second pressure sensor.

The first pressure sensor may be an ambient pressure sensor and the second pressure sensor is a pressure sensor in located a flow path of the apparatus.

In the non-therapy mode no flow of gases may be provided from the flow generator, so that the ambient pressure is the same as the pressure of the gases in the flow path.

At least one sensor may comprise a first temperature sensor and the further sensor comprises a second temperature sensor, and wherein the apparatus is configured to update the sensor calibration parameters of a first temperature sensor based on the output of a second temperature sensor.

The first temperature sensor may be an ambient temperature sensor and the second temperature sensor is a patient end temperature sensor in located near a patient end of conduit configured to be connected to the apparatus.

The first temperature sensor may be located at the same location as the second temperature sensor.

In the non-therapy mode the apparatus is configured to provide no power to a heater of the humidifier and/or a heater of a conduit for a predetermined period of time, so that the ambient temperature is the same as the temperature of the gases in the flow path.

The at least one sensor is located in one or more of:
a sensor module (optionally the sensor module is located between the flow generator and the humidifier),
the flow generator,
a location upstream of the flow generator,
a location downstream of the flow generator,
the humidifier,
a location upstream of the humidifier,
a location downstream of the humidifier,
a conduit configured to be connected to a gases outlet of the humidifier and convey the flow of gases to the user (optionally at a user end of the conduit near a patient interface)
a patient interface,
an ambient sensor,
a measuring chamber (optionally as part of the sensor module),
a humidification chamber inlet,
a humidification chamber outlet.

The apparatus may comprise at least one valve, the at least one valve configured be connected to a source of a supplemental gas, the valve configured to control a flow of supplemental gas.

The supplemental gas may be oxygen.

The flow of supplemental gas may be configured to be combined with ambient air and the combined supplemental gas and ambient air are provided to the flow generator.

The flow of supplemental gas may be configured to be added to the flow of gases generated by the flow generator.

The apparatus may be configured to is configured to operate the valve to control a supplemental gas concentration of the flow of gases provided to the user to at a therapy oxygen concentration.

The apparatus may comprise at least one patient oxygen saturation sensor, and wherein apparatus is configured to operate the valve to control a supplemental gas concentration of the flow of gases provided to the user based on an output of the at least one patient oxygen saturation sensor to reach a therapy patient oxygen saturation.

When the apparatus is updating parameters of the apparatus, the apparatus may be configured to operate the valve to prevent the flow of supplemental gas.

When the apparatus is configured to operate the valve to prevent the flow of supplemental gas, the concentration of the supplemental gas in the flow of gases may be assumed to be the concentration of the supplemental gas in ambient air.

The apparatus may comprise an alternative supply inlet, the alternative supply inlet configured to be connected to a source of a supplemental gas.

A flow of supplemental gas from the alternative supply inlet may be configured to be combined with ambient air and the combined supplemental gas and ambient air is provided to the flow generator.

A flow of supplemental gas from the alternative supply inlet may be configured to be added to the flow of gases generated by the flow generator.

Before the apparatus updates parameters of the apparatus, the apparatus may be configured to prompt a user (optionally via a user interface) to disconnect the source of a supplemental gas from the alternative supply inlet.

The at least one sensor may be an oxygen concentration sensor, optionally the oxygen concentration sensor comprises an ultrasonic sensor.

When no supplemental gas is provided as part of the flow of gases, the apparatus may be configured to determine an output of the oxygen concentration sensor indicative of an oxygen concentration of the flow of gases, wherein based on the output of the oxygen concentration sensor and an estimated oxygen ambient concentration the apparatus is configured to determine an oxygen concentration sensor calibration parameter.

The estimated oxygen ambient concentration may be about 19% to about 23% about 20.9%, or about 21%, or about 22%.

When supplemental gases are provided as the flow of gases, the apparatus may be configured to determine an output of the oxygen concentration sensor indicative of an oxygen concentration of the flow of gases, wherein based on the output of the oxygen concentration sensor and a predetermined oxygen concentration the apparatus is configured to determine an oxygen concentration sensor calibration parameter.

The predetermined oxygen concentration may be 100%.

No ambient air may be provided as part of the flow of gases.

The predetermined oxygen concentration may be entered by a user.

The user may be prompted to connect a supplementary source to the apparatus and indicate an oxygen concentration of the supplementary source.

The apparatus may be configured to run the flow generator at a predetermined flow rate, or a predetermined motor speed.

The apparatus may be configured to run the flow generator at a predetermined flow rate, or a predetermined motor speed after the apparatus has determined the oxygen concentration sensor calibration parameter.

The at least one sensor may be a flow rate sensor configured to measure a flow rate of the flow of gases.

During the non-therapy mode, the apparatus may be configured to stop the flow generator from generating the flow of gases and determine an output of the flow rate sensor indicative of the flow rate of the gases, wherein based on the output of the flow rate sensor and a predetermined no flow rate, the apparatus is configured to determine a flow rate sensor calibration parameter.

The predetermined no flow rate may be 0 LPM.

During the non-therapy mode, the apparatus may be configured to, while the flow generator is generating the flow of gases, determine an output of the flow rate sensor indicative of the flow rate of the gases, wherein based on the output of the flow rate sensor and a predetermined flow rate, the apparatus is configured to determine a flow rate sensor calibration parameter to be applied to outputs of the flow rate sensor.

The predetermined flow rate may be above 0 LPM, or about 10 LPM, or about 20 LPM, or about 30 LPM, or about 40 LPM or about 50 LPM, or about 60 LPM, or about 70 LPM.

When supplemental gases are provided as the flow of gases, the apparatus may be configured to determine an output of the humidity sensor indicative of a humidity of the flow of gases of gases, wherein based on the output of the humidity sensor and a predetermined humidity the apparatus is configured to determine a humidity sensor calibration parameter.

The predetermined humidity may be 0% relative humidity, or no absolute humidity.

The controller may be configured to determine a humidity sensor calibration parameter based on an output of another humidity sensor.

The humidity sensor and/or the other humidity sensor may comprise:
an ambient humidity sensor
a gases flow humidity sensor.

The at least one non-therapy mode may comprise a drying mode configured to dry the conduit.

When the apparatus is operating in the drying mode a heater of the conduit may be controlled while the flow generator provides gases at a predetermined flow rate.

When the apparatus is operating in the drying mode a heater of the humidifier may be controlled to a predetermined value (optionally the predetermined value is a predetermined power, and the predetermined power is less than about 5% or less than about 10% of a maximum power provided to the heater plate), or the heater plate is be deactivated during the drying mode.

The heater of the conduit may be controlled to a predetermined temperature at the end of the conduit or controlled to a predetermined duty cycle, or to a predetermined voltage, or to a predetermined current or to a predetermined power.

The predetermined duty cycle may be 100%.

The predetermined temperature may be greater than 45 degrees Celsius.

The drying mode may be configured to be operated for about 20 minutes to about 120 minutes, or about 90 minutes.

The drying mode may comprise controlling the flow generator to provide a predetermined flow generator output, wherein the flow generator output is a motor speed about 1000 RPM to about 3000 RPM or less than about 2000 RPM.

The drying mode may comprise controlling the flow generator to provide a predetermined flow rate, wherein the predetermined flow rate is about 5 litres/minute to about 20 litres/minute.

The drying mode may be configured to evaporate remaining condensate in the apparatus and/or patient breathing conduit and/or patient interface.

The non-therapy mode may be a warm up mode.

The non-therapy mode may be a standby mode.

When the apparatus is operating in the at least one therapy mode, therapy may be being provided to the user.

The at least one therapy mode may comprise:
a) a Continuous Positive Airway Pressure (CPAP) mode,
b) a Bubble Continuous Positive Airway Pressure (BCPAP) mode,
c) a Nasal High Flow (NHF) mode,
d) a Bilevel mode
e) any combination of a)-d).

When the apparatus is operating in the at least one non-therapy mode, no therapy may be being provided to the user.

The apparatus may be configured to automatically operate in the at least one non-therapy mode after completion of the at least one therapy mode.

The apparatus may be configured to update parameters of the apparatus at one or more of:
the end of non therapy mode,
the beginning of non therapy mode.

The apparatus may comprise a controller, wherein the controller is configured to control the flow generator and/or the humidifier in at least one therapy mode and at least one non-therapy mode, wherein while operating in the at least one therapy mode the apparatus is configured to provide therapy to a user,

The controller may be configured to update at least one parameter of the apparatus.

After the apparatus has updated parameters of the apparatus, the apparatus may be configured to transmit data to the device.

The data may comprise the updated parameters of the apparatus.

The apparatus may comprise at least one display (optionally as part of a display module).

The display may display information relating to the sensor calibration process.

A report may be generated based on information relating to the sensor calibration process

The information may comprise one or more of: the sensor error and whether the sensor is within tolerance or outside of tolerance the success or failure of the calibration and/or a resolution to the fault of the sensor.

In another aspect of the disclosure, there is provided a flow generator configured to generate a flow of gases.
a humidifier configured to be pneumatically connected to the flow generator and to humidify the flow of gases,
wherein the apparatus is configured to be connected to a conduit that conveys the flow of gases
wherein the apparatus is configured to operate in at least a therapy mode and a drying mode, wherein in the therapy mode the apparatus is configured to provide therapy to a user according to one or more therapy parameters, and in the drying mode at the heater of the humidifier is deactivated, and a heater of the conduit is activated while the flow generator provides gases at a predetermined flow rate and/or a predetermined motor speed,
wherein while operating in the at least drying mode (and optionally after a predetermined time), the apparatus is configured to:
   transmit data to a device, and then,
   update at least one sensor calibration parameters of at least one sensor (and optionally at least two sensors) of the apparatus.

It will be appreciated that any of the above statements may be combined with any one or more other statements.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

It should be understood that alternative embodiments or configurations may comprise any or all combinations of two or more of the parts, elements or features illustrated, described or referred to in this specification.

Some embodiments of this disclosure may also be said broadly to consist or comprised in the parts, elements and features referred to or indicated in the specification of the application, individually or collectively, and any or all combinations of any two or more said parts, elements or features, and where specific integers are mentioned herein which have known equivalents in the art to which this disclosure relates, such known equivalents are deemed to be incorporated herein as if individually set forth.

The term “comprising” as used in this specification means ‘including’. When interpreting each statement in this specification that includes the term ‘comprising’, features other than that or those prefaced by the term may also be present. Related terms such as ‘comprise’ and ‘comprises’ are to be interpreted in the same manner.

The term request when used in the context of a controller may refer to the controller sending a signal to a component to instruct the component to perform one or more actions.

As used herein the term ‘(s)’ following a noun means the plural and/or singular form of that noun.

As used herein the term ‘and/or’ means ‘and’ or ‘or’, or where the context allows both.

The disclosure discloses the foregoing and also envisages constructions of which the following gives examples only.

It will be appreciated that when a list set out, the disclosure includes any combination of items in the list.

### BRIEF DESCRIPTION OF THE DRAWINGS

Specific embodiments and modifications thereof will become apparent to those skilled in the art from the detailed description herein having reference to the figures that follow, of which:

FIG. 1 shows in diagrammatic form a breathing assistance apparatus.

FIG. 2 illustrates a sensing circuit board that may be used in a breathing assistance apparatus.

FIG. 3 is a first underside perspective view of the main housing of breathing assistance apparatus showing a recess inside the housing for the motor and/or sensor module sub-assembly.

FIG. 3A is a second underside perspective view of the main housing of the breathing assistance apparatus showing the recess for the motor and/or sensor module sub-assembly.

FIG. 4 is a perspective view of a breathing assistance apparatus.

FIG. 5 is a perspective view of the motor and/or sensor subassembly, underside of the main housing, and fixed elbow of the breathing assistance apparatus.

FIG. 6 is an exploded perspective view of components of the motor and/or sensor sub-assembly schematically showing by way of an arrow the gas flow path through the sub-assembly.

FIG. 7 is an underside view of a cover and sensing PCB of the motor and/or sensor sub-assembly showing the position of sensors.

FIG. 8 is a schematic gas flow path diagram for the filter module and the valve module, with the solid line arrows representing the flow

FIG. 8A is a schematic diagram of a system including an apparatus and breathing conduit.

FIGS. 9-18 show a user interface presenting a health enquiry on a breathing assistance apparatus.

FIGS. 19A-20B show flow diagrams of an apparatus operating in a therapy mode and a non-therapy mode.

FIG. 21 is a perspective view of a breathing assistance apparatus with a disinfection conduit.

FIG. 22 shows a layout of data including therapy data and apparatus data.

FIG. 23 show a flow diagram of an apparatus undertaking an action.

FIG. 24 is a flow diagram showing a system for providing breathing assistance or providing respiratory therapy to a patient.

FIG. 25 shows a flow diagram of an apparatus operating in a NHF mode and a drying mode.

FIG. 25A shows a flow diagram of an apparatus operating in a non-therapy mode and a drying mode.

FIG. 26 shows a flow diagram of an apparatus operating in a NHF mode and a warm up mode.

FIG. 27 shows a flow diagram of the apparatus operating in a non-therapy mode and updating parameters of the apparatus.

FIG. 28 shows a flow diagram of the apparatus operating in a non-therapy mode and updating sensor calibration parameters of the apparatus.

FIGS. 29-31 show flow diagrams of the apparatus updating parameters of the apparatus.

FIG. 32 shows a flow diagram of an example of determining a sensor calibration parameter.

FIG. 33 shows a flow diagram of an example of determining an oxygen concentration sensor calibration parameter.

FIG. 34 shows a flow diagram of an example of determining a flow rate sensor calibration parameter.

FIG. 35 shows a flow diagram of an example of determining a humidity sensor calibration parameter.

FIG. 36 shows a flow diagram of an example of the apparatus moving to a non-therapy mode after the completion of a therapy mode.

### DETAILED DESCRIPTION

A breathing assistance apparatus 10 provides therapy to a user based on one or more therapy modes. The therapy modes relate to a specific type of therapy for example (NHF, CPAP, NIV etc.). The therapy modes also comprise one or more therapy parameters of the apparatus specific to the type of therapy provided. For example, in a therapy mode which is providing Nasal High Flow therapy the therapy parameters may comprise a desired flow rate, and a desired dew point. Other types of high flow therapies are also contemplated e.g. tracheal high flow via an unsealed tracheal interface.

The breathing assistance apparatus may provide a number of different therapies according to associated therapy modes. For example the breathing assistance apparatus may provide Continuous Positive Airway Pressure therapy, Bubble Continuous Positive Airway Pressure (BCPAP) therapy, high flow therapy e.g. Nasal High Flow (NHF) therapy, BiLevel pressure therapy (for example NIV therapy). A user can select the appropriate mode of operation. The user will use an appropriate patient interface to enable provision of the selected therapy e.g. unsealed interfaces for high flow therapy. Alternatively, the breathing apparatus may be configured to provide only a single type of therapy.

During operation of the apparatus 10 the apparatus 10 may be configured to operate in one or more non-therapy modes. While the apparatus is operating in the non-therapy mode the user is not provided with therapy. The non-therapy modes may relate to modes of the apparatus 10 which relate to the transition into, or out of, a therapy mode (for example preparing the apparatus for a therapy mode, or to transition the apparatus from a therapy mode to storage, or power off). In the non-therapy modes, generally, no patient interface is engaged to a patient.

The therapy mode may be operated for a therapy session.

Example non-therapy modes may comprise a warm-up mode, a drying mode, a standby mode, a disinfection mode.

The non-therapy modes may be operated as a non-therapy process for a predetermined time (for example a drying mode), and/or until a desired sensor output is reached (for example a temperature requirement for a period of time in disinfection mode).

During operation the apparatus may undertake one or more actions, for example:
The apparatus may collect various data (for example usage data or other therapy compliance data, or data relating to parameters of therapy) while it is operating and transmit the data to a device.
The apparatus may receive a software package from a or the device (for example a firmware or software update).
The apparatus may receive therapy parameters from a or the device (for example an updated prescription).
The apparatus may receive update parameters of the apparatus (for example recalibrating one or more components of the device).
The device may be a device located locally with the apparatus (for example within the same household, or hospital—i.e. a tablet, or computer), or remotely (for example a server).

The apparatus may undertake the above actions during therapy however in this case the most up to date information is not provided to the device (if the therapy session hasn't been completed) or some actions for example a firmware update or an update in a parameter of the system may not be safe to undertake during therapy.

Undertaking the action during therapy may introduce a safety risk to the patient and/or health risk (i.e. interruption of therapy.) Further, if the action is one that may affect therapy parameters (for example receiving and applying therapy parameters) the therapy parameters may be updated in the middle of therapy session which may introduce a safety risk and/or a health risk.

If the action is undertaken on completion of the therapy mode there is a risk the user may completely power off the apparatus after therapy, early on in the operation of the apparatus in the non-therapy mode. If the apparatus is powered off during the action there is a risk of data corruption or incomplete transfer of data, or an error in applying a software package. Further some actions may not be possible while the apparatus is not being powered.

For example, an error in applying a software package may lead to an apparatus which has compromised performance or with certain features which do not work. This presents a health risk to the patient that is unacceptable.

In systems where therapy data may be uploaded at the conclusion of therapy, or after a time has elapsed after therapy, the user may power off the apparatus before the action is, or the apparatus may have to wait for a very long time to ensure the user will not power off the apparatus (which may lead to the issues described above).

Further, a user may be required to specially power on the apparatus, to undertake the action (for example to receive and apply a software package) Having to power on the apparatus specifically so that action can be undertaken may lead to inconvenience for the user, and if the user does not specifically instruct the apparatus undertake the action the action may not be undertaken (for example the software package may not be applied.)

The current disclosure introduces a predetermined time period operating in the non-therapy mode before initiating any of the actions. This reduces the probability of the user will completely power off the apparatus and mitigates the risk of the apparatus being powered off during the action.

This also ensures that the risk of the action interrupting or interfering with an important apparatus routine, and/or interrupting or interfering with therapy is mitigated.

The current disclosure may provide for a safe opportunity for the apparatus to initiate any of the actions when it is safe for the apparatus and/or the patient to undertake the action.

A safe opportunity for the apparatus may be when the risk to the apparatus hardware and/or software/firmware integrity is low. For example, when the risk of firmware or software updates being interrupted is low.

A safe opportunity for the patient may be when the patient is not connected to or using the apparatus for therapy while it is applying any changes, testing sensors, etc.

Further, after time operating in the non-therapy mode the risk of an alarm being raised, or an import routine needing to run which would interfere with the action needing to be run decreases. Beginning the action directly after the end of therapy (where a user may still need to be monitored for safety as they may not have removed their interface and still be connected to the apparatus) may introduce an increased risk of an alarm being raised, or an import routine needing to run.

Further, because a user may occasionally change their mind about starting a therapy session, they may power on the apparatus briefly before turning it off. If the apparatus attempts to undertake an action when the apparatus is turned on then this may be interrupted by the user turning the apparatus off.

The present disclosure also allows for an architecture where all data transfer to and from the apparatus 10, is initiated by the apparatus 10 and controlled by the apparatus 10. This may provide a security benefit as the apparatus 10 is no required to monitor connections from devices, and undertake actions in response to interrogation for a device.

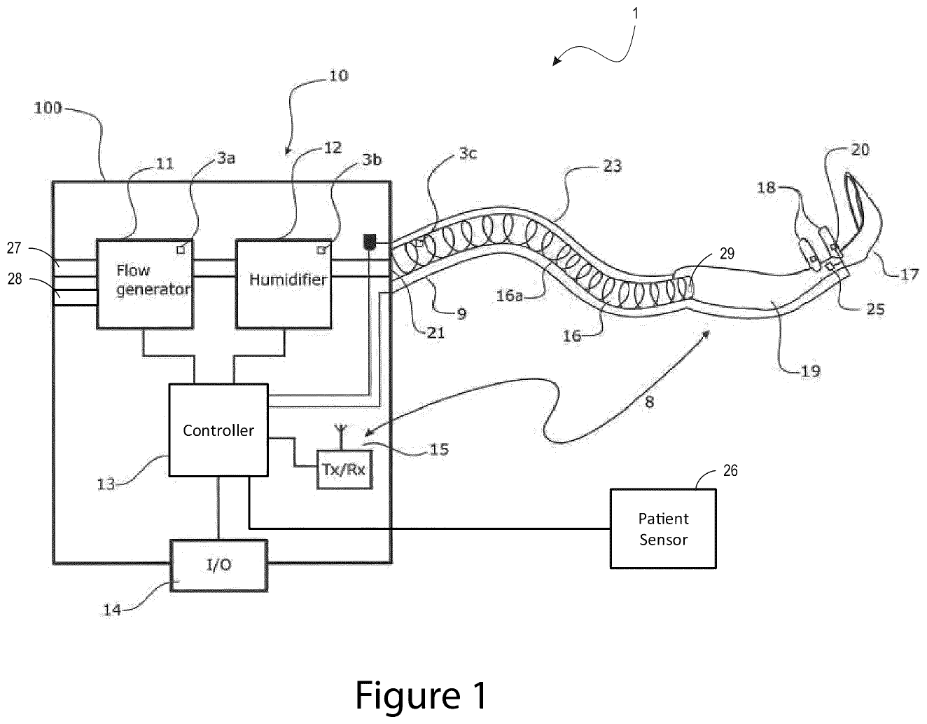

A breathing assistance apparatus 10 is shown in FIG. 1. The breathing assistance apparatus 10 can comprise a housing 100 that contains one or more of: a flow generator 11, which in some embodiments is in the form of a motor/impeller arrangement (for example, a blower), a humidifier 12 pneumatically connected to the flow generator 11, a controller 13, and a user interface 14 (comprising, for example, a display and input device(s) such as button(s), a touch screen, or the like).

The humidifier 12 can humidify the gases flow and/or heat the gases flow to an appropriate level. The controller 13 can be configured to control the humidifier 12 (for example by controlling at least a humidifier heater).

The humidifier 12 may comprise a humidification chamber 300. The humidification chamber 300 may be configured to be removed from the humidifier (for example for replacement, cleaning and/or refilling). Alternatively, the humidification chamber 300 may be non-removable from the humidifier.

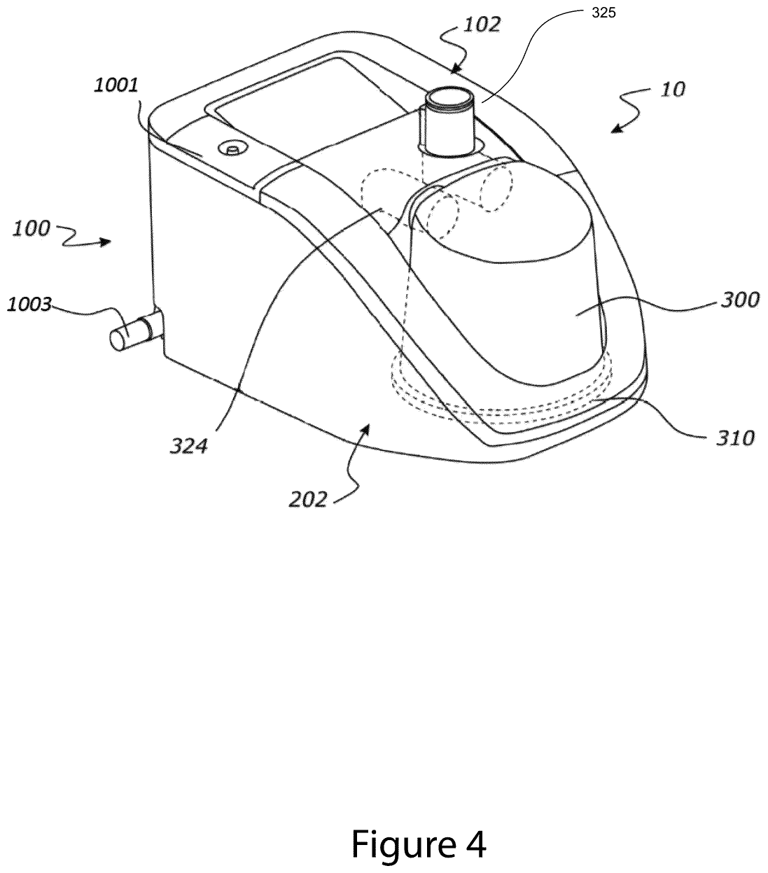

The humidifier 12 may comprise a humidifier heater 310 for example as a heater plate (see FIG. 4). The humidifier heater provides heat to the humidification chamber 300. The liquid in the humidification chamber 300 may be water or another liquid, and/or may comprise a mixture of one or more liquids (for example a mixture of water and a medicament.)

The controller 13 can be configured or programmed to control the operation of the breathing assistance apparatus 10. For example, the controller 13 can control components of the breathing assistance apparatus 10, including but not limited to: operating the flow generator 11 to create a flow of gas (gases flow) for delivery to a patient, operating the humidifier 12 (if present) to humidify and/or heat the generated gases flow, controlling a flow of oxygen into the flow generator blower, receiving user input from the user interface 14 for reconfiguration and/or user-defined operation of the breathing assistance apparatus 10, and outputting information (for example on the display) to the user.

The controller 13 may include one or more computer processors and associated non-transitory memory or storage mediums storing processor-executable instructions or code. The instructions, when executed by the one or more processors cause the respiratory therapy apparatus to affect the steps and processes described herein.

It will be appreciated that when the specification describes the apparatus 10 undertaking an action, it may be that the controller 13 is controlling one or more components of the apparatus 10 to undertake the action.

It will be appreciated the methods described herein can be executed by the controller (or another processor).

The term breathing assistance apparatus may be used interchangeably with respiratory assistance apparatus, or respiratory therapy apparatus or flow therapy apparatus.

The term breathing assistance system may be used interchangeably with respiratory assistance system, or respiratory therapy system, or flow therapy system.

The term current flow rate may refer to a measurement of a flow rate which has been presently made (for example at a current time step). It will be appreciated that the term current flow rate is not limited to the latest flow rate determination and could include recently made flow rate determinations (for example from a previous time step or the most recent flow rate determination), and/or a filtered flow rate determination made based on a series of past measurements (which may optionally include signal filtering and/or processing).

The methods described herein may be embodied as software or a software module as part of control software (for example computer-readable instructions) that are stored in the controller (or associated memory) and executed by the controller (and/or an associated processor).

In the context of receiving therapy the user is a patient, however in the context of interacting with the apparatus (for example interacting with a user interface) the user can be one or more of a patient, healthcare professional (for example a clinician), or anyone else interested in using the apparatus.

As used herein, a “gases flow” can refer to any flow of gases that may be provided by the breathing assistance apparatus, such as a flow of ambient air, a flow comprising substantially 100% oxygen, a flow comprising some combination of ambient air and oxygen, and/or the like.

A breathing conduit 16 is coupled at one end to a gases outlet 21 in the housing 100 of the breathing assistance apparatus 10. The breathing conduit 16 is coupled at another end to a patient interface 17 such as a non-sealed nasal cannula with a manifold 19 and nasal prongs 18. Additionally, or alternatively, the breathing conduit 16 can be coupled to a face mask, a nasal mask, a nasal pillows mask, an endotracheal tube, a tracheostomy interface, and/or the like.

In some configurations, the interface 17 may be a sealing interface for example when the apparatus is providing a CPAP therapy.

A breathable conduit may be provided between the breathing conduit 16 and the patient interface 17.

In some embodiments, a different conduit type may be connected to the gases outlet 21, for example a disinfection conduit in the disinfection mode.

The gases flow that is generated by the breathing assistance apparatus 10 may be humidified and delivered to the patient via the breathing conduit 16 and the patient interface 17.

The breathing conduit 16 can have a heater 16a to heat the gases flow passing through to the patient. The heater 16a can be under the control of the controller 13. In at least one configuration, the heater 16a is a heater wire. The breathing conduit 16 and/or patient interface 17 can be considered part of the breathing assistance therapy system. The breathing assistance system 1 may comprise the breathing assistance apparatus 10, breathing conduit 16, and patient interface 17.

The controller 13 can control the flow generator 11 to generate a gases flow at the desired flow rate (for example a therapy flow rate). The controller 13 can also control a supplemental oxygen inlet to allow for delivery of supplemental oxygen.

The controller 13 can also control a humidifier heater in the humidifier 12 and/or the heater 16a in the breathing conduit 16 to heat the gas to a desired temperature for a desired level of therapy and/or level of comfort for the patient.

The controller 13 can be provided with or can determine a suitable target temperature of the gases flow. The controller 13 may control the humidifier heater of the humidifier 12 and/or the heater 16a of the breathing conduit based on one or more suitable target temperature(s) of the gases flow.

The heater 16a in the breathing conduit 16 may be controlled by the controller 13 to reach a desired temperature. The desired temperature may be, or be based on, one or more temperature set points, and/or one or more humidity set points (for example a therapy humidity).

The humidifier heater of the humidifier 12 may be controlled by the controller 13 to reach a desired temperature. The desired temperature may be, or be based on, one or more temperature set points, and/or one or more humidity set points. The desired temperature may be a therapy parameter.

The controller 13 may control the heater 16a in the breathing conduit 16 and/or the humidifier heater of the humidifier 12 to the desired temperature by closed loop control based on the output of one or more sensors.

The one or more temperature set points may relate to one or more therapy parameters of the apparatus for therapy (for example a dew point or temperature of the gases), or be provided in the memory of the apparatus (for example a predetermined temperature).

The one or more therapy parameters in high flow therapy mode e.g. nasal high flow mode (NHF mode) may comprise any combination of:
a therapy flow rate of the gases provided to the user,
a therapy humidity level (for example a relative or absolute humidity, or a dew point)
a therapy oxygen concentration provided to the user,
a therapy concentration of an auxiliary gas provided to the user,
a therapy temperature of the gases provided to the user (for example).

The one or more therapy parameters in BCPAP mode may comprise any combination of:
a therapy flow rate of the gases provided to the user,
a therapy humidity level (for example a relative or absolute humidity, or a dew point)
a therapy oxygen concentration provided to the user,
a therapy concentration of an auxiliary gas provided to the user,
a therapy temperature of the gases provided to the user.

The one or more therapy parameters in CPAP mode may comprise any combination of:
a therapy humidity level (for example a relative or absolute humidity, or a dew point)
a therapy oxygen concentration provided to the user,
a therapy temperature of the gases provided to the user.

a therapy concentration of an auxiliary gas provided to the user,
a therapy level of pressure support (for example a CPAP pressure) provided to the user
a therapy PEEP pressure provided to the user.

The one or more therapy parameters in Bilevel mode may comprise any combination of:
a therapy humidity level (for example a relative or absolute humidity, or a dew point)
a therapy oxygen concentration provided to the user,
a therapy temperature of the gases provided to the user.
a therapy concentration of an auxiliary gas provided to the user,
a therapy IPAP/EPAP pressure (inspiratory positive airway pressure/expiratory positive airway pressure) provided to the user.

The therapy temperature may comprise a therapy temperature at the chamber outlet and/or a therapy temperature at the end of the breathing conduit.

The therapy humidity may be at the chamber outlet or at the end of the breathing conduit.

The therapy humidity level may be a dew point of about 37 degrees Celsius, or an absolute humidity of above about 38 mg H2O or about to 44 mgH2O.

This humidity level can cause condensate during use, and it is important to dry this the breathing conduit 16 before re-use of the conduit. This is particularly required in the homecare environment where tubes are reused (for example between 7-14 days). In hospitals tubes are often replaced for new patients but the same patient may re-use the same tube. To reduce infection risk and risk of pathogen growth, breathing conduit is dried to remove moisture including liquid (for example by operating a drying mode as described in more detail below.)

Given the high flow provided during NHF mode, and corresponding humidity this may increase the amount of condensate which forms, which may further increase the importance of a drying mode as described in more detail below.

The user may enter one or more therapy types associated with a therapy mode via the user interface.

The user may enter one or more therapy parameters via the user interface.

The desired temperatures may be at end of the breathing conduit 16, at the patient interface, at the gases outlet, a humidification chamber outlet, at any sensor of the apparatus, and/or any combination thereof.

The one or more temperature set points may comprise one or more of:
a desired dew point (for example a temperature indicative of a desired humidity),
a predetermined dew point,
a predetermined temperature,
a desired temperature.

In some embodiments the controller 13 may control the heater 16a of the breathing conduit 16 based on a desired temperature of the gases at the patient interface and/or a desired temperature at the end of the breathing conduit 16.

The apparatus may be powered by an external power source (for example a wired connection with an electrical grid).

In some embodiments, the apparatus may be powered by at least one battery. The battery may be located in the housing of the apparatus, and/or attached externally to the housing of the apparatus. In some embodiments, the battery is removable. Alternatively the battery is non removable.

As shown for example in FIG. 4, the oxygen inlet port 28 includes a valve 1003 through which a pressurized gas may enter the respiratory therapy apparatus 10. The valve can control a flow of oxygen into the respiratory therapy apparatus 10. The valve can be any type of valve, including a proportional valve or a binary valve.

The source of oxygen can be an oxygen tank or a hospital oxygen supply. Medical grade oxygen is typically between 95% and 100% purity. Oxygen sources of lower purity can also be used. Examples of valve modules and filters are disclosed in U.S. Provisional Application No. 62/409,543, titled “Valve Modules and Filter”, filed on Oct. 18, 2016, and U.S. Provisional Application No. 62/488,841, titled “Valve Modules and Filter”, filed on Apr. 23, 2017, which are hereby incorporated by reference in their entireties.

The breathing assistance apparatus 10 can measure and control the oxygen content of the gas being delivered to the patient, and therefore the oxygen content of the gas inspired by the patient.

The breathing assistance apparatus 10 may provide high flow therapy, in which the high flow rate of gas delivered meets or exceeds the peak inspiratory demand of the patient.

Operation sensors 3a, 3b, 3c, such as flow, temperature, humidity, and/or pressure sensors can be placed in various locations in the breathing assistance apparatus 10. Additional sensors (for example, sensors 20, 25) may be placed in various locations on the breathing conduit 16 and/or patient interface 17 (for example, there may be a temperature sensor 29 at or near the end of the inspiratory tube).

The respiratory therapy apparatus 10 may have a communications module 15 to enable the controller 13 to receive signals 8 from the sensors and/or to control the various components of the breathing assistance apparatus 10, including but not limited to the flow generator 11, humidifier 12, heater 16a, humidifier heater, or accessories or peripherals associated with the breathing assistance apparatus 10. Additionally, or alternatively, the communications module 15 may deliver data to a remote server or enable remote control of the respiratory therapy apparatus 10 or respiratory therapy system 1.

The communications module may comprise a transmitter, receiver and/or transceiver.

The communications module 15 may act as a network interface.

The communications module 15 may use one or more communication protocols known in the art, for example Wi-Fi, Bluetooth, Zigbee, cellular (3G, 4G, or 5G, etc.).

The communications module may comprise a number of separate transmitters, receivers and/or transceiver for each, or for a group of communication protocol(s).

The communications module 15 may be configured to transmit data and receive data from one or more devices (for example a server) as described in more detail below.

In some embodiments, one or more leak or blockage events, or alarms (as described in more detail below) may be transmitted to one or more servers and/or devices (for example a computer, phone or tablet). Additional information (for example the time, duration, or severity) associated with the event or alarm may be additionally transmitted to the server and/or device.

As described above, the breathing assistance apparatus 10 can measure and control the oxygen content of the gas being delivered to the patient. Oxygen may be measured by placing one or more gas composition sensors (such as an ultrasonic transducer system) after the oxygen and ambient air have been mixed. The measurement can be taken within the respiratory therapy apparatus 10, the patient breathing conduit 16, the patient interface 17, or at any other suitable location.

The oxygen concentration measured in the apparatus may be equivalent to the fraction of delivered oxygen (FdO2) and may be substantially the same as the oxygen concentration the patient is breathing, the fraction of inspired oxygen (FiO2), and as such the terms may be seen as equivalent.

Oxygen concentration may also be measured by using flow rate sensors on at least two of the ambient air inlet conduit, the oxygen inlet conduit, and the patient breathing conduit to determine the flow rate of at least two gases. By determining the flow rate of both inlet gases or one inlet gas and one total flow rate, along with the assumed or measured oxygen concentrations of the inlet gases (about 20.9% for ambient air, about 100% for oxygen), the oxygen concentration of the final gas composition can be calculated. Alternatively, flow rate sensors can be placed at all three of the ambient air inlet conduit, the oxygen inlet conduit, and the breathing conduit to allow for redundancy and testing that each sensor is working correctly by checking for consistency of readings. Other methods of measuring the oxygen concentration delivered by the breathing assistance apparatus 10 can also be used.

The breathing assistance apparatus 10 can include a patient sensor 26, such as a pulse oximeter or a patient monitoring system, to measure one or more physiological parameters of the patient, such as a patient's blood oxygen concentration (for example blood oxygen saturation (SpO2)), heart rate, respiratory rate, perfusion index, and provide a measure of signal quality. The sensor 26 can communicate with the controller 13 through a wired connection or by communication through a wireless transmitter on the sensor 26. The sensor 26 may be a disposable adhesive sensor designed to be connected to a patient's finger. The sensor 26 may be a non-disposable sensor (i.e. a re-useable sensor). Sensors that are designed for different age groups and to be connected to different locations on the patient are available and can be used with the breathing assistance system 1. The pulse oximeter can be attached to the patient, typically at their finger, although other places such as an earlobe are also an option. The pulse oximeter can be connected to a processor in the respiratory therapy apparatus 10 and can constantly provide signals indicative of the patient's blood oxygen saturation. The patient sensor 26 can be a hot-swappable device, which can be attached or interchanged during operation of the breathing assistance apparatus 10. For example, the patient sensor 26 may connect to the breathing assistance apparatus 10 using a USB interface or using wireless communication protocols (such as Bluetooth®).

When the patient sensor 26 is disconnected during operation, the breathing assistance apparatus 10 may continue to operate in its previous state of operation for a defined time period. After the defined time period, the breathing assistance apparatus 10 may trigger an alarm, transition from automatic mode to manual mode, and/or exit control mode (e.g., automatic mode or manual mode) entirely. The patient sensor 26 may be a bedside monitoring system or other patient monitoring system that communicates with the breathing assistance apparatus 10 through a physical or wireless interface.

The breathing assistance apparatus 10 may comprise or be in the form of a high flow therapy apparatus.

High flow therapy as discussed herein is intended to be given its typical ordinary meaning as understood by a person of skill in the art which generally refers to a breathing assistance apparatus delivering a targeted flow of humidified respiratory gases via an intentionally unsealed patient interface with flow rates generally intended to meet or exceed inspiratory flow of a patient. Typical patient interfaces include, but are not limited to, a nasal or tracheal patient interface. Typical flow rates for adults often range from, but are not limited to, about fifteen litres per minute to about sixty litres per minute or greater. Typical flow rates for paediatric patients (such as neonates, infants and children) often range from, but are not limited to, about one litre per minute per kilogram of patient weight to about three litres per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (THF), among other common names.

For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient, ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may, in some configurations, deliver gases to the patient at a flow rate of between about 1 LPM and about 100 LPM, or at a flow rate in any of the sub-ranges outlined above. Gases delivered may comprise a percentage of oxygen. In some configurations, the percentage of oxygen in the gases delivered may be between about 20% and about 100%, or between about 30% and about 100%, or between about 40% and about 100%, or between about 50% and about 100%, or between about 60% and about 100%, or between about 70% and about 100%, or between about 80% and about 100%, or between about 90% and about 100%, or about 100%, or 100%.

High flow therapy may be effective in meeting or exceeding the patient's inspiratory flow, increasing oxygenation of the patient, and/or reducing the work of breathing.

High flow therapy may be administered to the nares of a patient and/or orally, or via a tracheostomy interface.

High flow therapy may generate a flushing effect in the nasopharynx such that the anatomical dead space of the upper airways is flushed by the high incoming gases flow. This can create a reservoir of fresh gas available for each and every breath, while reducing re-breathing of nitrogen and carbon dioxide. Meeting inspiratory demand and flushing the airways is additionally important when trying to control the patient's FdO2. High flow therapy can be delivered with a non-sealing patient interface such as, for example, a nasal cannula. High flow therapy may slow down respiratory rate of the patient. High flow therapy may provide expiratory resistance to a patient.

High flow therapy may be used to treat patients with obstructive pulmonary conditions e.g. COPD, bronchiectasis, dyspnea, cystic fibrosis, emphysema and/or patients with respiratory distress or hypercapnic patients.

The term “non-sealing patient interface” (i.e. unsealed patient interface) as used herein can refer to an interface providing a pneumatic link between an airway of a patient and a gases flow source (such as from flow generator 11) that does not completely occlude the airway of the patient. A non-sealed pneumatic link can comprise an occlusion of less than about 95% of the airway of the patient. The non-sealed pneumatic link can comprise an occlusion of less than about 90% of the airway of the patient. The non-sealed pneumatic link can comprise an occlusion of between about 40% and about 80% of the airway of the patient. The airway can include one or both nares of the patient and/or their mouth. For a nasal cannula the airway is through the nares.

CPAP therapy may comprise providing gases to a user at a continuous positive pressure (and optionally one or more therapy parameters as described in more detail above.)

BCPAP therapy may comprise providing gases to a user at a therapy flow rate (and optionally one or more therapy parameters as described in more detail above.)

Bilevel therapy may comprise providing gases to a user at a therapy flow rate (and optionally one or more therapy parameters as described in more detail above.)

A sealed interface, may be used when the apparatus is provided CPAP, Bilevel or BCPAP therapy,

The flow generator 11 can be or comprises a blower module. The blower module may comprise at least one blower 11′ configured to generate said flow of gases.

The flow generator 11 can include an ambient air inlet port 27 through which ambient room air can be entrained into the blower. The breathing assistance apparatus 10 may also include an oxygen inlet port 28 leading to a valve through which a pressurized gas may enter the flow generator 11. The valve can control a flow of oxygen into the flow generator 11. The valve can be any type of valve, including a proportional valve or a binary valve.

The blower 11′ can operate at a motor speed of greater than about 1,000 RPM and less than about 8,000 RPM, greater than about 2,000 RPM and less than about 10,000 RPM, or between any of the foregoing values. The blower 11′ can mix the gases entering the blower 11′ through the inlet ports (for example, the ambient air inlet port 27 and/or an oxygen inlet port 28). Using the blower 11′ as the mixer can decrease the pressure drop relative to systems with separate mixers, such as static mixers comprising baffles.

It will be appreciated that another supplemental gas may be provided instead of oxygen, and for example the oxygen inlet port 28 could be a supplemental gas inlet port, and the valve may be configured to control the flow of the supplemental gas.

The breathing assistance apparatus may further comprise a gas composition sensor. The gas composition sensor may be the sensor described below (for example the ultrasonic transducer configuration).

The breathing assistance apparatus 10 comprises a flow rate sensor. The flow rate sensor may be configured to measure a flow rate of the flow of breathable gas to a patient.

The controller 13 may comprise one or more processors. The processors may be configured with computer-readable instructions.

The controller 13 may comprise at least one memory element. The memory element may be configured to store said computer-readable instructions.

The memory element may be non-transitory computer readable medium.

The controller 13 may be a microprocessor or an ASIC, FPGA or a combination of ICs or microprocessors or other suitable components and/or architectures.

The breathing assistance apparatus may comprise at least one display module, configured to display an alarm output.

The breathing assistance apparatus may comprise at least one audible module configured to emit an audible alarm.

In some embodiments the at least one audible module may comprise a speaker.

The display module may comprise at least one display (for example a liquid crystal display (LCD), or a light emitting diode (LED) display, although it will be appreciated any display technology may be used).

The display module may be configured to receive inputs to the system (for example as a touch screen) and therefore be at least part, or display part of the user interface 14.

The display module may be configured to be an input/output (I/O) module. For example, the display module may be configured to receive inputs from a user and provide outputs to a user (for example as part of, or to display part of the user interface 14).

The display module may communicate with the controller 13. In some embodiments the display module may provide information to the controller 13 (for example set points). In some embodiments the display module may receive information from the controller 13 (for example alarms, sensor outputs, and/or other calculated variables.)

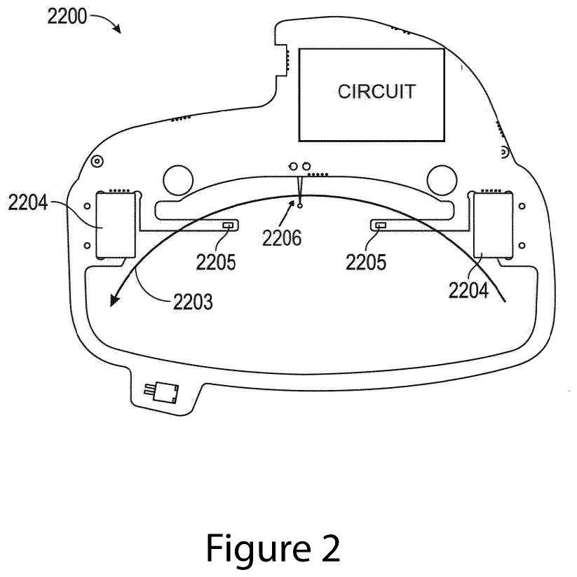

With additional reference to FIG. 2, a sensing circuit board 2200 is shown that can be implemented in the breathing assistance apparatus 10. The sensing circuit board 2200 can be positioned in a sensor chamber such that the sensing circuit board 2200 is at least partially immersed in the flow of gases. The flow of gases may exit the blower 11′ through a conduit and enter a flow path in the sensor chamber. At least some of the sensors on the sensing circuit board 2200 can be positioned within the flow of gases (shown in direction by arrow 2203) to measure gas properties within the flow. After passing through the flow path in the sensor chamber, the gases can exit to the humidifier 12 described above.

The sensing circuit board 2200 can be a printed sensing circuit board (PCB). Alternatively, the circuit on the board 2200 can be built with electrical wires connecting the electronic components instead of being printed on a circuit board. At least a portion of the sensing circuit board 2200 can be mounted outside of a flow of gases. The flow of gases can be generated by the flow generator 11 described above. The sensing circuit board 2200 can comprise ultrasonic transducers 2204. The sensing circuit board 2200 can comprise one or more thermistors 2205. The thermistors 2205 can be configured to measure a temperature of the gases flow. The sensing circuit board 2200 can comprise a thermistor flow rate sensor 2206. The sensing circuit board 2200 can comprise other types of sensors, such as humidity sensors (including humidity only sensors to be used with a separate temperature sensor and combined humidity and temperature sensors), sensors for measuring barometric pressure, sensors for measuring differential pressure, and/or sensors for measuring gauge pressure. The thermistor flow rate sensor 2206 can comprise a hot wire anemometer, such as a platinum wire, and/or a thermistor, such as a negative temperature coefficient (NTC) or positive temperature coefficient (PTC) thermistor. Other non-limiting examples of the heated temperature sensing element include glass or epoxy-encapsulated or non-encapsulated thermistors. The thermistor flow rate sensor 2206 can be configured to measure the flow rate of the gases by being supplied with a constant power, or by being maintained at a constant temperature or a constant temperature difference between the sensor and the flow of gases.

Positioning the one or more of thermistors 2205 and/or the thermistor flow rate sensor 2206 downstream of the combined blower and mixer means that the sensor readings will be dependent on the heat supplied to the gases flow by the blower. Furthermore, immersing at least part of the sensing circuit board and sensors in the flow path can increase the accuracy of measurements. Relative to sensors that are not immersed, sensors that are immersed in the flow are more likely to be subject to the same conditions, such as temperature and pressure, as the gases flow. Therefore, these immersed sensors may provide a better representation of the gases flow characteristics.

The sensing circuit board 2200 can comprise ultrasonic transducers, transceivers, or other sensors to measure properties of the gases flow, such as gas composition or concentration of one or more gases within the gases stream. Any suitable transducer, transceiver, or sensor may be mounted to the sensing circuit board 2200 as will be appreciated. In this configuration, the gas composition sensor is an ultrasonic transducer that employs ultrasonic or acoustic waves for determining gas concentrations.

The ultrasonic transducers may be an ultrasonic transducer pair arranged opposite each other in the sensor chamber, axial to the direction of flow. The ultrasonic transducer pair may be configured to determine flow rate using time of flight measurements.

Some examples of flow therapy apparatuses are disclosed in International Application No. PCT/NZ2016/050193, titled “Flow Path Sensing for Flow Therapy Apparatus”, filed on Dec. 2, 2016, and International Application No. PCT/IB2016/053761, titled “Breathing Assistance Apparatus”, filed on Jun. 24, 2016, which are hereby incorporated by reference in their entireties.

The apparatus 10 may comprise an elbow 325 which is configured to connect to a breathing conduit 16 (and for example provide the gases outlet 21). The elbow 326 may comprise one or more sensors.

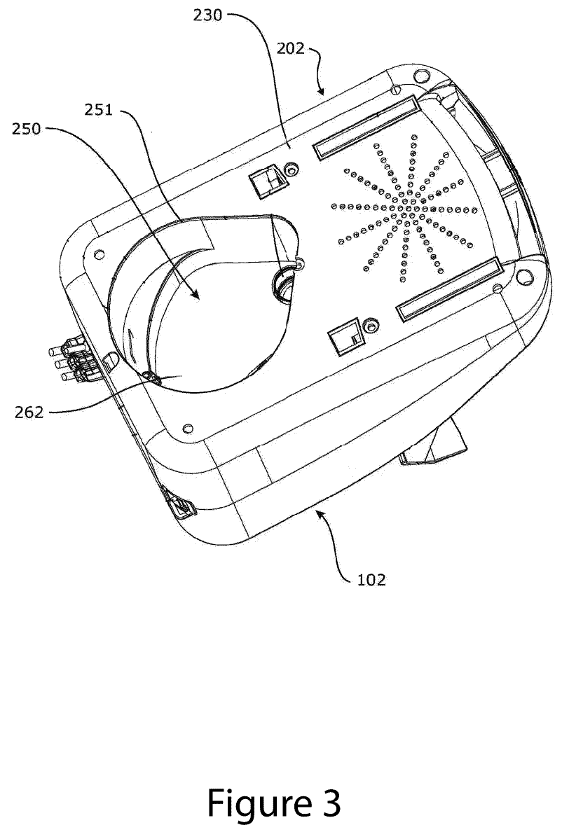

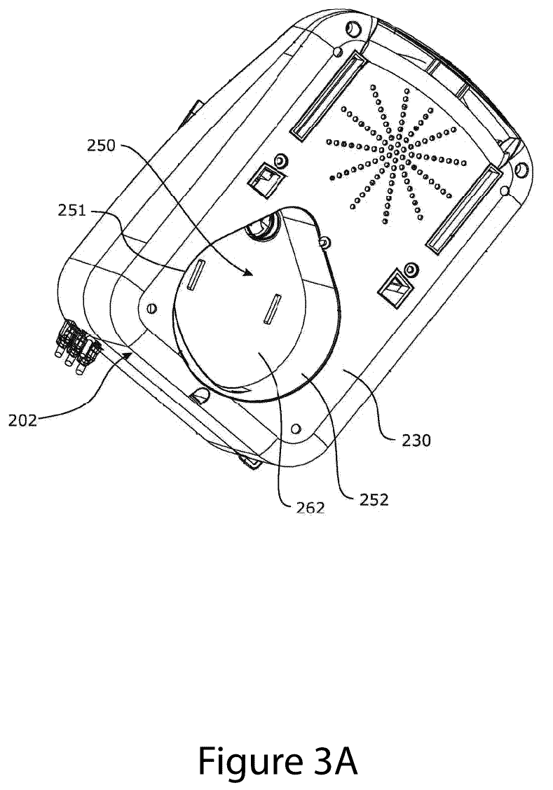

A configuration of a breathing assistance apparatus 10 is illustrated in FIGS. 3 to 7. As shown for example in FIG. 4, the breathing assistance apparatus comprises a housing 100. The housing 100 has a housing upper chassis 102 and a housing lower chassis 202.

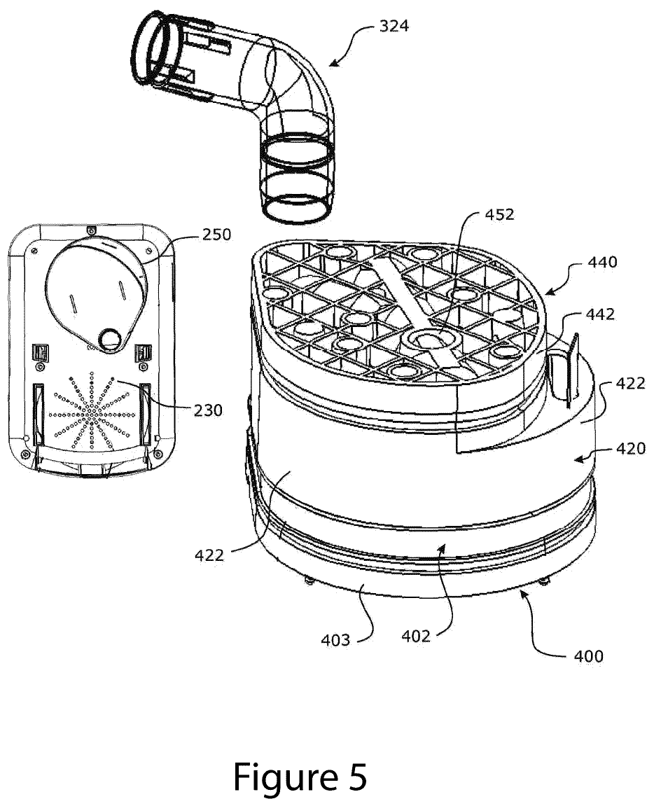

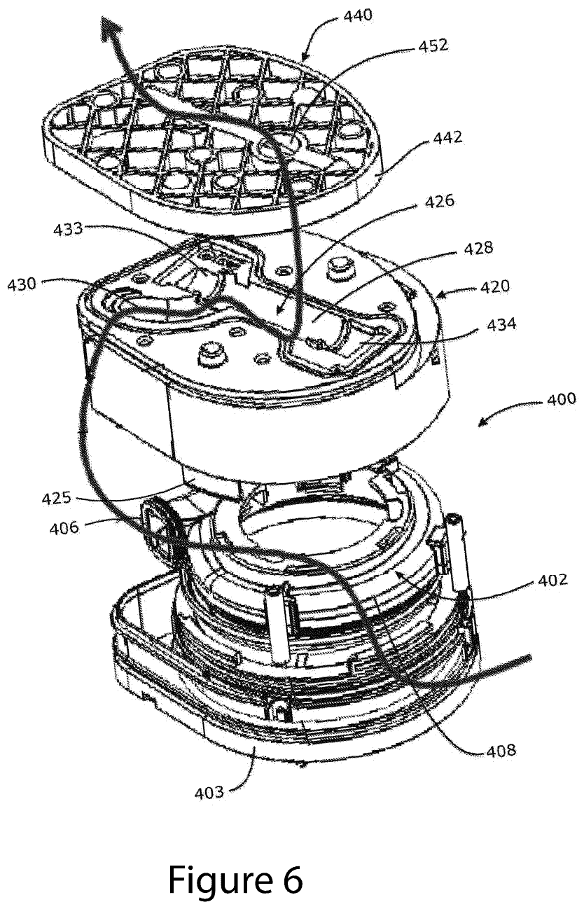

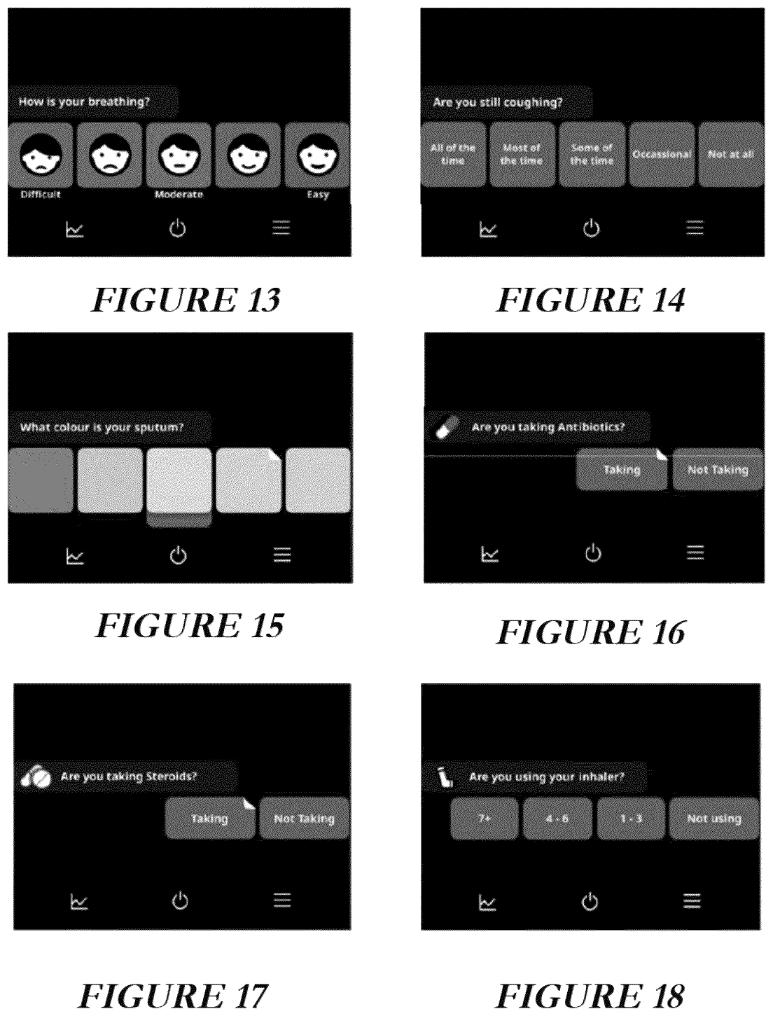

As shown in FIGS. 3 and 3A, the lower chassis 202 has a motor recess 250 for receipt of a removable or non-removable motor and/or sensor module 400 which is shown in FIGS. 13 to 15 and will be described in further detail below. A recess opening 251 is provided in the bottom wall 230 adjacent a rear edge thereof, for receipt of a removable or non-removable motor/sensor module 400 which is shown in FIGS. 5 and 6 and will be described in further detail below.

FIGS. 5 to 7 show the motor and/or sensor module or sub-assembly 400 in greater detail. As discussed above, the lower chassis 202 comprises a recess 250 for receipt of the motor and/or sensor module 400. The flow generator may comprise the motor and/or sensor module or sub-assembly 400.

In the form shown in FIGS. 5 to 7, the motor and/or sensor module 400 comprises a stacked arrangement of three main components: a base 403 of the sub-assembly 400 (on which is positioned the motor 402), an outlet gas flow path and sensing layer 420 positioned above the base 403, and a cover layer 440. The base 403, the sensing layer 420, and the cover layer 440 assemble to form a sub-assembly housing that has a shape that is complementary to that of the recess 250 so that the sub-assembly 400 can be received in the recess 250. The base 403 is configured to close the recess opening 251 when the sub-assembly 400 is positioned in the recess 250. The sub-assembly 400 may be maintained in position in the recess in any suitable way such as with fasteners, clips, or a quick release arrangement for example, or fixed in a non-removable manner.

The sensing layer comprises a gas flow path with one or more sensors, the gas flow path arranged to deliver gas to the outlet port of the housing.

The motor 402 has a body 408 that defines an impeller chamber that contains an impeller. The motor 402 could be any suitable gas blower motor, and may for example be a motor and impeller assembly of the type described in published PCT specification WO2013/009193. The contents of that specification are incorporated herein in their entirety by way of reference.

A gases outlet 406 is in fluid communication with a gases inlet of the outlet gas flow path and sensing layer 420, which is stacked on top of the motor. This layer 420 comprises a body 422 which comprises a plurality of mounting legs 425 that can be inserted into a plurality of mounting slots (not shown) of the base 403 to secure the body 422 to the base 403. In one configuration, the body 422 defines a gas flow path that couples the gases outlet 406 with the gases inlet of the gas flow path and sensing layer 420.

The body 422 defines a lower portion 426 of a sensing and gas flow path. The cover layer 440 has a body 442 that defines the upper portion 446 of the sensing and gas flow path, with the shape of the upper and lower portions 426, 446 corresponding substantially to each other.

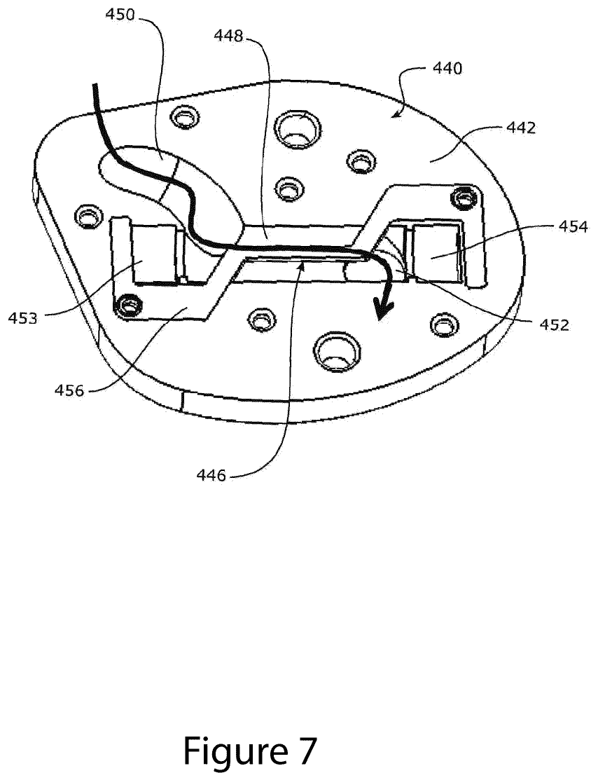

As shown in FIGS. 6 and 7, the gas flow path comprises a linear elongate gas flow portion 428, 448. The inlet is in fluid communication with a tangential entrance portion 430, 450 of the gas flow path, which is located at or adjacent an entrance end of the linear elongate portion 428, 448 of the gas flow path. Recesses 433, 453 and 434, 454 may be provided at opposite ends of the linear elongate portion of the gas flow path.

A gas flow outlet port 452 extends vertically through the body 442 of the cover layer 440 and is located at or adjacent an opposite exit end of the linear elongate portion 428, 448 of the gas flow path. The gas outlet port 452 is in fluid communication with an upper portion of the motor recess 250, which in turn is in fluid communication with the gas flow passage. Again, due to the wall 252 and ceiling 262 configuration of the recess 250, if there is gas leakage from the motor/sensor module 400, that will be vented to atmosphere rather than entering the portion of the housing 100 that contains the bulk of the electronics and control equipment. The recess 250 may comprise spacer(s), such as lugs that protrude downwardly from ceiling 262 as shown in FIG. 15, to maintain a suitable spacing for gas flow from the gas outlet port 452 and the ceiling of the recess 262.

It can be seen from FIG. 7 that that at least part of the gas flow path through and out of the motor and/or sensing module 400 has a tortuous or sinuous configuration. For example, the direction of gas flow travel through the elongate portions 428, 448 is generally opposite to the direction of gas flow travel from the gas outlet port 452 to the entrance of the gas flow passage through elbow 324. The tortuous or sinuous configuration may increase residence time of the gas in the gas flow path and therefore improves sensing.

As shown in FIGS. 6 and 7, the cover layer 440 comprises a sensing printed circuit board (PCB) 456. The cover layer 440 may also comprise one or more temperature sensors such as thermistors that sit in the elongate portion 428, 448 of the gas flow path. One sensor will measure gas temperature and the other can act as a redundant temperature sensor. Alternatively, one of the thermistors could be used as a reference flow rate sensor (e.g. via use as a constant-temperature thermistor), and the measured temperatures could be used to determine the gas flow rate through the portion 428, 448 of the gas flow path. The one or more temperature sensors may be located on a portion of the sensing PCB 456 that faces the gases flow. The sensing PCB 456 may additionally comprise other sensors including but not limited to pressure sensors, humidity sensors, and dew point sensors.

One or both electronics boards 272 will be in electrical communication or coupled with the sensors to process information received from the sensors and operate the apparatus 10 based on the information received from the sensors.

In an alternative configuration, the motor/impeller unit may be provided remotely from the apparatus 10. In that configuration, the module received in the recess 250 may only comprise a gas flow path and various sensors, to deliver gases to the fixed elbow 324 and thereby to the humidification chamber 300. In an alternative configuration, the module received in the recess 250 may only comprise the motor and a gas flow path, but no sensors.

In another alternative configuration the motor and/or sensor module 400 may not be removable from the recess 250, but instead may be permanently mounted therein.

Some configurations may provide the benefit of isolating the electrical/electronic components from the gases in the gases flow.

The flow path is compact and has reduced turns/sharp turns which reduces flow separation and reduces resistance to flow.

The arrangement of the motor and flow path provides another layer of isolation because of the wall arrangement.

Having a modular motor and/or sensor module enables the various parts of the module to be taken apart if needed for cleaning and/or servicing and/or replacement of components.

In some embodiments, there are no leak paths in the motor and/or sensor module.

The motor and/or sensor module may be a potential leak point, however, a leak in that region would result in the oxygen venting to atmosphere or into the liquid chamber.

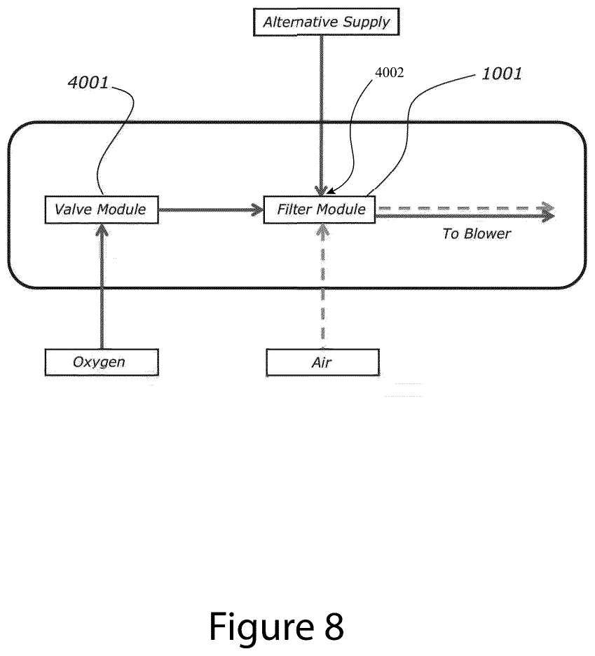

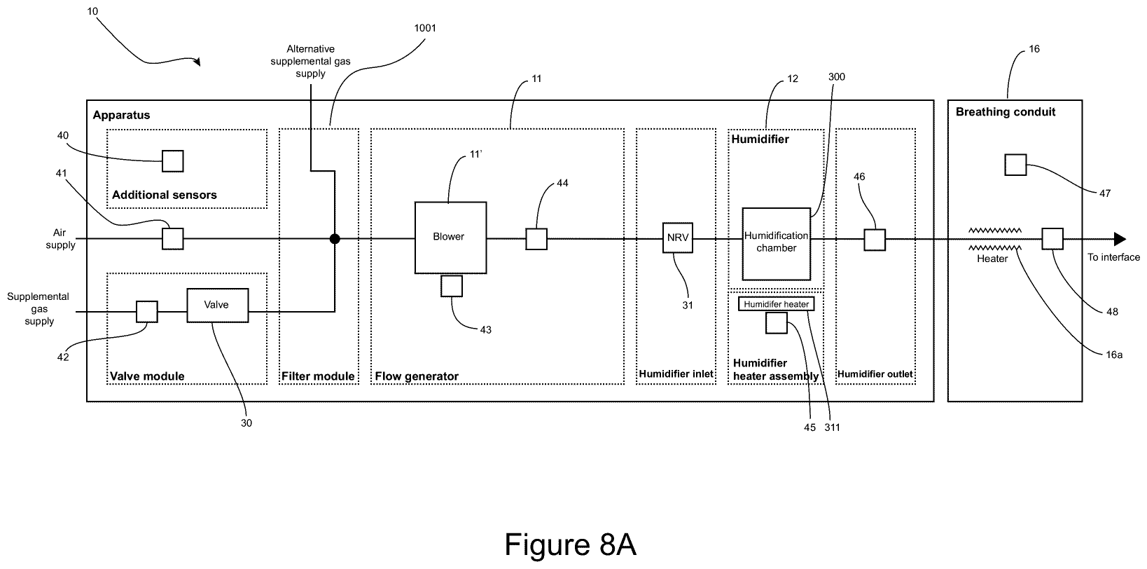

For example, as shown in FIG. 8, the apparatus 10 may comprise a valve module 4001 that controls the flow of oxygen and/or other gases (for example another supplemental gas) entering the gas flow path of the apparatus 10, and enables the apparatus 10 to regulate the proportion of oxygen (or another supplemental gas) entrained in the airflow. The valve module is formed as a modular unit for ease of manufacture, assembly, servicing, or replacement. For example, in the event of malfunction, routine maintenance, or future upgrade/improvement.

The valve module 4001 may be configured to operate to control the oxygen concentration (or other supplemental gases concentration) of the gases provided to the user to at a therapy oxygen concentration.

The valve module 4001 may comprise one or more filters located a) upstream of the valve, b) downstream of the valve, or c) both upstream and downstream of the valve.

The apparatus 10 may comprise a filter module 1001, which may comprise a filter.

The filter modules 1001 and valve modules 4001 described herein may provide varying gas flow paths for the apparatus. For example, the valve module may control the flow of oxygen entering the gas flow path of the apparatus, via the valve module and filter module. Alternatively, the valve module may be bypassed by means of direct connection of an alternative oxygen source to the filter module via an alternative supply inlet. This may be practical in circumstances where a user may wish to manually adjust the oxygen supply (i.e. by a wall-supply rotameter).

It will be appreciated that the apparatus may be provided with a different supplemental gas than oxygen.

It will be appreciated that the filter modules and the valve modules described herein may be used separately in apparatuses for delivering a flow of gas. Alternatively, the filter and the valve module may be used together as a filter and valve assembly for improved functionality.

In the configurations shown, the apparatus 10 receives oxygen by at least one of the following:
via the valve module 4001 (for automatic oxygen regulation by the apparatus), or
via the alternative gases inlet provided on the top of the filter (allowing attachment of a manually adjustable oxygen supply-such as a wall supply regulated by a regulator).

The apparatus 10 may comprise a manifold. The manifold may be located on the housing. The manifold may provide one or more of: the supplemental gases inlet (for example an oxygen inlet), the alternative gases inlet, and/or the air inlet.

The manifold may provide the oxygen, alternative gases, and/or ambient air to the valve module 4001, filter module 1001, and/or the blower 11′ of the flow generator 11.

The oxygen inlet or alternative gasses supply inlet may be provided on a side of the manifold.

The manifold may allow excess oxygen to overflow to the ambient environment, and/or may allow oxygen to overflow to the ambient environment if the blower is off and oxygen is continually supplied. This prevents accumulation of 02 in the housing.

FIG. 8A shows a schematic view of the apparatus 1. FIG. 8A shows various locations of sensors 40, 41, 42, 43, 44, 45, 46, 47 and 48 in the system which are described in more detail below.

A gases flow path may be provided from one or more inlets through the filter module 1001 to the patient interface via the generator 11, humidifier 12 and breathing conduit 16.

As described above the apparatus 10 may receive air (for example ambient air via an air inlet port 27). The air inlet may comprise at least one air inlet sensor. The at least one air inlet sensor 41 may comprise a temperature sensor and/or a humidity sensor (for example an absolute humidity sensor and/or a relative humidity sensor).

As described above the apparatus 10 may receive a supplemental gas (for example oxygen via oxygen inlet port 28). The supplemental gas inlet may comprise at least one supplemental gas inlet sensor 42. The valve module 4001 as described above may be configured to operate to control the flow of supplemental gas (for example oxygen).

The at least one supplemental gas inlet sensor 42 may be part of the valve module 4001 or separate. In FIG. 8A, the at least one supplemental gas inlet sensor 42 is shown as part of the valve module 4001 and located upstream of the valve 30. In some configurations, the sensor oxygen inlet sensor 42 may be provided separate from the valve module 4001, and upstream, or downstream of the valve module 4001.

The at least one supplemental gas inlet sensor 42 may comprise at least a pressure sensor. The pressure sensor may be configured to measure the pressure of the supplemental gas supply.

As described above the apparatus 10 may receive supplemental gas via an alternative supply inlet.

The apparatus 1 may comprise one or more additional sensors 40. The additional sensors may be located within the apparatus (for example within the housing) and/or exposed to the ambient environment. The additional sensors may comprise a pressure sensor (for example an ambient pressure sensor). In some embodiments, the one or more additional sensors 40 are located at or near the valve module 4001.

As described above, the filter module 1001 receives the gases from the supplemental gas from the supplemental gas inlet and/or the alternative supply inlet, and the air from the air inlet.

As shown in FIG. 8A, the flow generator 11 is pneumatically connected to the filter module. The flow generator 11 comprises a blower 11′ (as described in more detail elsewhere in the specification.) The flow generator 11 may comprise the motor and/or sensor module 400 as described in more detail above.

The flow generator 11 may comprise at least one blower sensor 43 configured to measure a characteristic of the blower. The at least one blower sensor 43 may be configured to measure a characteristic of the motor of the blower. The at least one blower sensor 43 may comprise a motor speed sensor.

The at least one blower sensor 43 may measure an electrical characteristic of the blower (for example the motor of the blower).

The at least one blower sensor 43 may be located as part of the blower, or located away from the blower on for example a control board (for example when the at least one blower sensor 43 measures an electrical characteristic of the blower).

The flow generator may also comprise at least one sensor 44 located downstream of the blower 11′ (as for example as shown in FIG. 8A. In some configurations, the at least one sensor 44 is provided upstream of the blower. The at least one sensor 44 may be provided as part of the sensor module 400 (as described in more detail above). The at least one sensor 44 may comprise one or more of: at least one temperature sensor, at least one flow rate sensor (for example one or more ultrasonic transducers as described above), at least one pressure sensor (for example an absolute pressure sensor and/or a differential pressure sensor configured to measure the pressure in the gases flow path relative to ambient), at least one humidity sensor.

The sensor 3a as described above may be the blower sensor 43 and/or sensor 44.

As shown in FIG. 8A, the apparatus 10 may also comprise at least one non return valve (NRV) 31 in the humidifier inlet. The humidifier inlet may for example be fixed elbow 324 as described above.

The humidifier 12 as described in more detail above is pneumatically connected to the flow generator (for example via at least the fixed elbow 324). The humidifier heater may comprise at least one humidifier heater sensor 45. The at least one humidifier heater sensor 35 may be a temperature sensor and/or a humidifier heater power sensor configured to measure the power provided to the humidifier heater. The humidifier heater power sensor may be located away from the heater (for example on a control board).

As shown in FIG. 8A, the apparatus 10 may also comprise at least one humidifier outlet sensor 46 located in the humidifier outlet. The humidifier outlet may be the elbow 325. The humidifier outlet sensor 46 may be one or more temperature sensors.

The sensor 3b as described above may be the humidifier heater sensor 45 and/or humidifier outlet sensor 46. Similarly, the sensor 3c may be the humidifier outlet sensor 46.

As described above, a breathing conduit 16 comprises a heater 16a of the breathing conduit 16. The breathing conduit 16 may comprise at least one breathing conduit sensor 48 (for example sensor 29 as described above). The at least one breathing conduit sensor 48 may be located at a patient end of the conduit 16. The at least one breathing conduit sensor 48 may be a temperature sensor.

A heater 16a of the breathing conduit 16 power sensor 47 may also be provided to measure the power provided to the heater 16a of the breathing conduit. The power sensor 47 may be located away from the breathing conduit 16 (for example on a control board).

The various configurations described are exemplary configurations only. Any one or more features from any of the configurations may be used in combination with any one or more features from any of the other configurations.

As another example, while the motor and/or sensor sub-assembly recess is described as being in the underside of the housing, it could alternatively be in the rear, side, front, or top of the housing. The air and/or oxygen inlets may also be positioned differently as required.

As another example, rather than the humidification chamber 300 and chamber bay being configured so that the humidification chamber 300 is inserted into and removed from the chamber bay from a front of the housing, the configuration could be such that the humidification chamber 300 is inserted into and removed from the chamber bay from a side, rear, or top of the housing.

As another example, while the filter modules are described as being inserted into the housing from above and the valve modules inserted into the housing from below, either or both of those components could be inserted into any suitable part of the housing, such as an upper part, lower part, side part, front part, or rear part.

The filter module and valve module are described with reference to a breathing assistance apparatus that can deliver heated and humidified gases to a patient or user.

The filter module and/or valve module may alternatively be used with an apparatus that does not require a humidifier and therefore does not require the humidification chamber 300. For example, it will be appreciated that the configuration that isolates the motor and gas flow path from the electrical and electronic components has broad applications in other types of gas delivery apparatuses.

The apparatus may be powered on (i.e. to a powered on state) by connecting the apparatus to a power source (i.e. a battery or electrical connection) and/or by one or more inputs of the apparatus (for example a power switch and/or an input on the user interface)

The apparatus may be powered off (i.e. to a powered off state) by disconnecting the apparatus from a power source (i.e. a battery or electrical connection) and/or by one or more inputs of the apparatus (for example a power switch and/or an input on the user interface)

The apparatus 10 may present a health enquiry comprising one or more queries, when the apparatus is in a non-therapy mode. In some embodiments the non-therapy mode in which the heathy enquiry is presented may be a warm up mode, and/or a drying mode (as described in more detail below).

The apparatus 10 may present one or more health enquiry to a user.

The health enquiry may comprise one or more comprising one or more user queries relating to one or more health parameters.

Each query comprises a plurality of user input elements via which user inputs are received as answers to the user queries.

The apparatus 10 may health enquiry may be that as disclosed in WO 2021/090184 (PCT application number PCT/IB2020/060335) which is hereby incorporated by reference.

The apparatus 10 may present the health enquiry at the beginning of the non-therapy mode.

The apparatus 10 may present the health enquiry:
on startup of the apparatus, or
at the beginning of the non-therapy mode, or
when the user is prompted to enter one or more therapy parameters of the breathing assistance apparatus (optionally via a therapy control screen), or
when the user begins to enter one or more therapy parameters of the breathing assistance apparatus (optionally via a therapy control screen), or
once the user has initiated therapy (optionally via a therapy control screen), or
when manually activated by a user,
or any combination of the above.

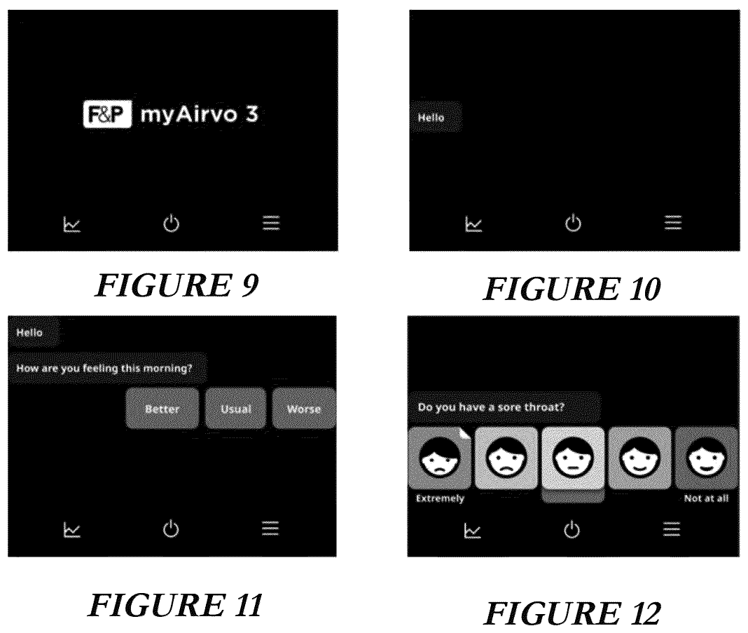

FIGS. 9 to 18 show a user interface presenting a health enquiry on a breathing assistance apparatus. The plurality of questions and a plurality of potential answers are displayed.

The plurality of questions may relate to one or more health parameters of a patient.

FIG. 9 shows a booting screen that has a graph user touch element, a power user touch element, and a menu user touch element. For example, the booting screen can be a first screen that the patient sees when the patient turns on the breathing assistance apparatus, as described herein. The graph user touch element can enable a presentation of a data graph for various therapy parameters or patient health parameters when selected, as described herein, whether this presentation occurs on same page or on different page of the user interface. The power user touch element can be used to power on or power off (for example as described above) or restart the breathing assistance apparatus when selected, as described herein.

FIG. 10 shows an introductory screen after the booting screen. The introductory screen displays a hello message (or some other introductory or welcoming message).

FIG. 11 shows a general feeling screen that presents a query (e.g., request for user health information, health query) inquiring about a general feeling of the patient at a specific time of day.

FIG. 12 shows a sore throat screen that presents a query, similar to FIG. 15.

However, unlike the query and the potential answers of FIG. 15, the query and the potential answers of FIG. 12 relate to a throat soreness parameter of the patient. The query of FIG. 12 comprises multiple possible answers each with an associated icon. The icons are colour coded relative to the patient condition associated with the answer, and have faces with expressions related to the patient condition associated with the answer (as described in more detail below).

FIG. 13 shows a breathing screen that presents a query, similar to FIGS. 11 and 12. However, unlike the queries and the potential answers of FIGS. 11 and 12, the query and the potential answers of FIG. 13 relate to a breathing parameter of the patient.

FIG. 14 shows a coughing screen that presents a query similar to FIGS. 11 to 13.

FIG. 15 shows a sputum color screen that presents a query similar to FIGS. 11 to 14.

FIG. 16 shows an antibiotic use screen that presents a query similar to FIGS. 11 to 15.

FIG. 17 shows a steroid use screen that presents a query similar to FIGS. 11 to 16.

FIG. 18 shows an inhaler use screen that presents a query similar to FIGS. 11 to 17.

A health provider may set one or more patient baselines relating to a question and/or a health parameter.

When the corresponding question (or question relating to a health parameter) is displayed the baseline may be displayed to the user. The baseline may be indicated by a graphical element (for example the folded comed in FIGS. 12, 15, 16, 17) or via highlighting.

In some embodiments, the health enquiry (and/or one or more questions and answers) may be presented when the apparatus enters operation in the non-therapy mode (for example when the apparatus enters a drying mode). This may allow for the user to complete the health enquiry before the predetermined time operating in the non-therapy mode has elapsed. Therefore, when the action is undertaken (as described in more detail below) the answers to the health enquiry (and/or one or more answers to the questions) may be available for transmission to the device.

In some embodiments, the health enquiry (and/or one or more questions and answers) may be presented in a first non-therapy mode (for example a warm up mode) and transmitted to the device (as discussed in more detail below) in a second non-therapy mode (for example a drying mode).

The data relating to the health enquiry (and/or one or more questions and answers) may be packaged into a single package or there may individual packages for therapy data and questionnaire answer data.

The health enquiry (and/or one or more questions and answers) may be stored in the memory of the apparatus after being collected in the first non-therapy mode before being transmitted during operation in the second non-therapy mode (for example after a predetermined time as described in more detail below.)

The apparatus may operate in first non-therapy mode before the therapy session (for example a warm up mode) and a second non-therapy mode after the therapy session (for example a drying mode after the therapy session.

In some embodiments, during a non-therapy mode a therapy summary screen is displayed. The therapy summary screen may include aspects of the therapy data (described below in more detail).

As described above the apparatus may be configured to operate in at least one therapy mode and at least one non-therapy mode.

When operating in a non-therapy mode, the apparatus may display information relating to the non-therapy on the display.

The information may be one or more of: the type of non-therapy mode, an indication that the non-therapy mode is active, a time remaining in the non-therapy mode, a warning not to use the apparatus.

In the therapy mode the apparatus is configured to provide therapy to a user.

The therapy mode may comprise:
a) a Continuous Positive Airway Pressure (CPAP) mode,
b) a Bubble Continuous Positive Airway Pressure (BCPAP) mode,
c) a Nasal High Flow (NHF) mode,
d) a BiLevel pressure mode (for example an NIV mode) where pressure is controlled between an IPAP and EPAP,
e) any combination of a)-d).

Each therapy mode may have one or more associated therapy parameters (as described in more detail above) of the apparatus for example a therapy flow rate of the gases, a therapy pressure support level, a therapy temperature of the gases, therapy humidity of the gases, a therapy temperature at the end of the breathing conduit, etc).

Each therapy mode may have associated software configured to be executed by the apparatus 10 (for example by the controller 13) to control the apparatus to provide the specific therapy.

Generally, only one type of therapy mode is provided at once (i.e. CPAP and NHF cannot be delivered concurrently).

In each therapy mode, the user may be provided with a different user interface, or different options for input for the apparatus.

In each therapy mode, the apparatus may comprise a different control scheme (for example a pressure control scheme for CPAP mode and a flow control scheme for NHF mode).

In each therapy mode, the apparatus may have one or more alarm conditions. One or more alarms can be raised if the alarm conditions are met. The alarm conditions may be based on the specific therapy mode (i.e. the specific therapy being provided) and/or one or more fault conditions which may arise during therapy.

The apparatus is also configured to operate in one or more non-therapy mode. In the non-therapy mode no therapy is provided to a user.

The non-therapy modes may comprise a drying mode, a warm up mode, a standby mode and/or a disinfection mode.

In some embodiments, when the apparatus is operating in the at least one non-therapy mode, the flow generator is activated and is generating a flow of gases. In some embodiments, the when the apparatus is operating in the at least one non-therapy mode, the flow generator is controlled to a predetermined motor speed, and/or to achieve a predetermined flow rate.

In some configurations, the predetermined flow rate may be provided by controlling the motor speed of the flow generator. This may be by, for example, a look up table or formula defining the relationship between flow rate and motor speed. In non-therapy modes this approach may allow for easier control of the flow generator as no patient will be connected, and the resistance to flow may be known (i.e., in disinfection mode), or considered constant (i.e., in drying mode and/or disinfection mode).

In some configurations, to control the flow generator (for example to provide the predetermined flow rate and/or a predetermined motor speed) the apparatus may use feedback control based on one or more sensors, e.g. for control to a motor speed a motor speed sensor or a current/voltage sensing unit that monitors current or voltage to the motor may be used. For control to a flow rate a flow rate sensor may be used (for example an ultrasonic sensor and/or a thermistor as described elsewhere in the specification).

In some embodiments, when the apparatus is operating in the at least one non-therapy mode, the flow generator is configured to provide a flow rate of gases less than the therapy flow rate.

In some embodiments, when the apparatus is operating in the at least one non-therapy mode, the flow generator is configured to provide a flow rate less than about 50%, or less than about 25%, or less than about 10%, of the therapy flow rate.

In some embodiments, when the apparatus is operating in the at least one non-therapy mode the humidifier (optionally a heater of the humidifier) is activated. In some embodiments, when the apparatus is operating in the at least one non-therapy mode the humidifier is configured to humidify the flow of gases.

In some embodiments, when the apparatus is operating in the at least one non-therapy mode, the humidifier is configured to humidify the flow of gases at a humidity less than the therapy humidity.

In some embodiments, when the apparatus is operating in the at least one non-therapy mode, the humidifier is configured to humidify the flow of gases less than about 50%, or less than about 25%, or less than about 10%, of the therapy humidity (for example an absolute or relative humidity, and/or a dew point).

In some embodiments, when the apparatus is operating in the at least one non-therapy mode a heater of the conduit, is activated and is configured to heat the flow of gases in the breathing conduit.

In some embodiments, when the apparatus is operating in the at least one non-therapy mode, the heater of the conduit is configured to heat the flow of gases to an end of conduit temperature less than the therapy end of conduit temperature.

In some embodiments, when the apparatus is operating in the at least one non-therapy mode, the heater of the conduit is configured to heat the flow of gases less than about 50%, or less than about 25%, or less than about 10%, of the therapy temperature (for example a therapy temperature at the end of the conduit, or a therapy humidity as a dew point).

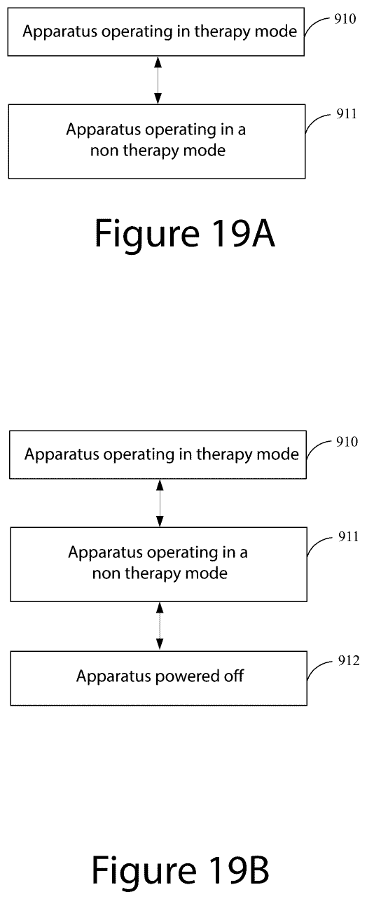

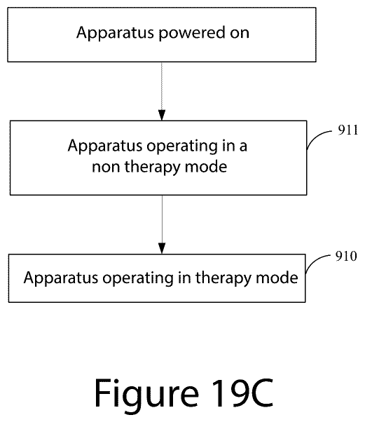

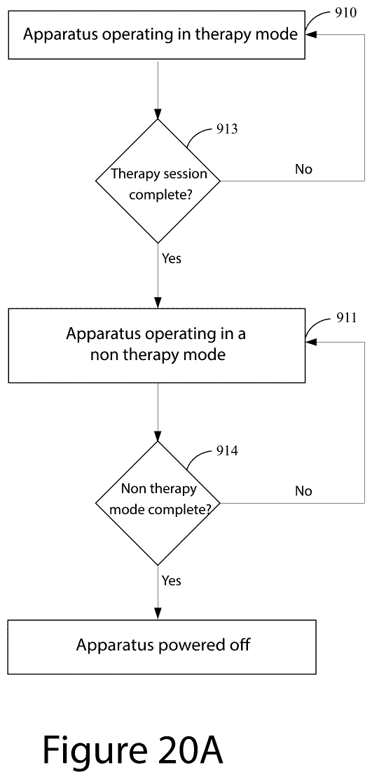

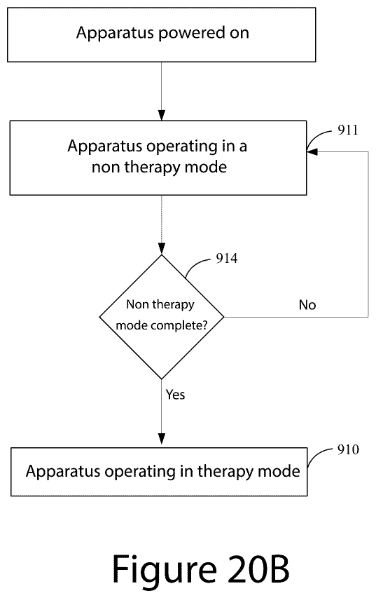

As illustrated in FIG. 19A, the apparatus may be configured to change between operation in the therapy mode 910 and operation in the non-therapy mode 911. The change between operation in the therapy mode 910 and operation in the non-therapy mode 911 may be based on one or more triggers for example indicating the therapy session is complete 913 as shown in FIG. 20A.

In some embodiments, for example as shown in FIG. 19B, the apparatus operating in the non-therapy mode 911 may act as a transition from the apparatus operating in a therapy mode 910 to the apparatus being powered off 912. The transition from operating in a therapy mode 910 to the apparatus being powered off 912 may be important as directly powering off the apparatus from the therapy mode may lead to damage to the apparatus or one or more components of the system. In this case the non-therapy mode may be for example a drying mode configured to dry the breathing conduit of the apparatus so it can be safely powered off.

As shown in FIG. 20A the apparatus may transition from operating in the therapy mode 910 to operation in the non-therapy mode 911 when the therapy session is complete. In some embodiments, the apparatus may be configured to automatically operate in the at least one non-therapy mode after completion of the at least one therapy mode. Alternatively, on completion of therapy, the user may be presented with a prompt on the user interface to transition to operation to the non-therapy mode.

The apparatus may transition to a second non-therapy mode in response to a trigger. The trigger may be elapsed time or may be a manual input (for example an input via the user interface).

The apparatus may determine the therapy session is complete when the user enters an end of therapy command.

The end of therapy command may be generated by an input via the user on the user interface.

The end of therapy command may be generated by detection that the patient interface has been removed from the user.

The end of therapy command may be generated by detection that the patient interface has been removed from the user for a predetermined amount of time.

Detection that the patient interface has been removed from the user can be performed according to the method(s) described in PCT publication no. WO 2020/178746 which is hereby incorporated by reference in its entirety.

Detection that the patient interface has been removed from the user may be based on estimates of flow conductance/flow conductance changes, or any other suitable method.

The end of therapy command may be generated by detection that the patient interface 17 has been disconnected from the breathing conduit 16 (optionally for a predetermined amount of time).

Detection that the patient interface has been removed may be made based on detecting that a user's breathing has ceased (optionally for a period of time.)

The user may be required to confirm the end of therapy command (via for example a user interface).

Detecting that a user's breathing has ceased may be based on one or more of:
a) the output of a patient interface sensor located in the patient interface,
b) a flow rate sensor located in the apparatus,
c) a pressure sensor located in the apparatus
any combination of a)-c).

Detection that the patient interface has been removed may be made based on a change in conductance of the flow path (optionally for a predetermined period of time).

As shown in FIG. 20A, the apparatus will power off when the non-therapy mode has been completed. The non-therapy mode may be completed based on time and/or one or parameters of the apparatus (for example a sensor) reaching a desired value.

The apparatus may operate in more than one non-therapy mode before the apparatus is powered off (i.e. from a powered on state to a powered off state).

The apparatus may be powered off automatically at the completion of the therapy mode, or by input from a user. In some embodiments, the user may be unable to power off the apparatus until the non-therapy mode has been completed.

In some embodiments, for example as shown in FIG. 19C, the apparatus operating in the non-therapy mode 911 may act as a transition from the apparatus being powered on to the apparatus operating in a therapy mode 910. The transition from a powering on the apparatus to operating in a therapy mode may also be important, as the apparatus may not be able to immediately operate in a therapy mode and provide adequate therapy to the user (for example not being able to provide the gases at the therapy parameters). In this case the non-therapy mode may be for example a warm up mode configured to warm up the apparatus before it begins operating in the therapy mode.

As shown in FIG. 20B the apparatus may transition from operating in the non-therapy mode 911 to operation in the therapy mode 910 when the non-therapy mode is complete (as discussed in more detail above). In some embodiments, the apparatus may be configured to automatically operate in the at least one therapy mode before operating of the at least one therapy mode. Alternatively, on completion of non-therapy mode, the user may be presented with a prompt indicating the apparatus is ready for therapy and the user may enter an input on the user interface to transition to operation to the therapy mode.

The apparatus may operate in more than one non-therapy mode after the apparatus is powered on (i.e., from a powered off state to a powered on state).

As discussed above, the non-therapy mode may be a drying mode. The apparatus may operating in the drying mode to dry the breathing conduit.

The drying mode reduces risk of pathogen growth in the humid post-therapy environment and can extend the safe lifetime of breathing conduits. Drying mode is operated for at least 30 mins, but preferably at least 90 mins in order to minimize risk of pathogen (i.e. microbial) growth in the conduit (i.e. tube).

The drying mode may be of particular importance in a NHF system, as due to the high flow provided to the user and corresponding humidity, a large amount of condensate may form during use.

The drying mode may be the drying process described in PCT publication no. WO 2006/126900 which is hereby incorporated by reference.

Operating the apparatus in a drying mode is important after the apparatus finishes operating in a therapy mode. The drying mode allows for any condensate in the breathing conduit after therapy to be removed.

The apparatus may operate in the drying mode after operating in a therapy mode, and the therapy mode is complete (for example as shown in FIGS. 19B and 20A and described in more detail above).

In some configurations, ozone gas may be provided to the gases flow path while in the drying mode.

While the apparatus is operating in the drying mode the heater of the breathing conduit is controlled while the flow generator provides gases at a predetermined flow rate.

When the apparatus is operating in the drying mode a heater of the humidifier is deactivated.

When the apparatus is operating in the drying mode a heater of the breathing conduit is controlled to a predetermined temperature at the end of the breathing conduit or controlled to a predetermined duty cycle, or to a predetermined voltage, or to a predetermined current or to a predetermined power.

The predetermined duty cycle may be 100%.

The predetermined temperature is greater than 45 degrees Celsius.

The drying mode may be configured to be operated for about 20 minutes to about 120 minutes, or about 90 minutes.

If the user attempts to turn the apparatus off before completion of the drying mode, the apparatus may display a message to the user that the drying mode has not completed and/or prevent the user from powering off the apparatus or powering off the apparatus without further confirmation.

The drying mode comprises controlling the flow generator to provide a predetermined flow generator output, wherein the flow generator output is a motor speed about 1000 RPM to about 3000 RPM or less than about 2000 RPM.

The drying mode comprises controlling the flow generator to provide a predetermined flow rate, wherein the predetermined flow rate is about 5 litres/minute to about 20 litres/minute.

The drying mode is configured to evaporate remaining condensate in the apparatus and/or breathing conduit and/or patient interface.

In some embodiments, when the apparatus is operating in the drying mode the apparatus presents a message to the user notifying the user not to wear the patient interface 17.

As discussed above, the non-therapy mode may be a warm up mode. The apparatus may operate in the warm up mode to prepare the apparatus for the therapy mode.

Operating the apparatus in a warm up mode before a therapy mode may be important to ensure when treatment is commenced the apparatus is providing the flow of gases at the therapy parameters for the therapy mode. Having the apparatus activate the heater of the humidifier and/or the heater of the breathing conduit in advance of a therapy mode helps the apparatus provide the flow of gases at the therapy parameters for the therapy mode when the apparatus beings to operate in the therapy mode.

In some embodiments, in the warm up mode the heater of the humidifier is activated.

In some embodiments, in the warm up mode the heater of the humidifier and/or the heater of the breathing conduit are activated.

In some configurations, in the warm up mode, the heater of the humidifier and/or the heater of the breathing conduit are activated at 100% power, for example 100% duty cycle.

The apparatus may operate in warm up mode when the apparatus is powered on (i.e. from a power off state to a power on state).

The warm up mode may comprise controlling a heater of the breathing conduit to control a temperature at the end of the conduit to a desired temperature.

The desired temperature at the end of the conduit may be based on one or more therapy parameters of the apparatus.

The desired temperature at the end of the breathing conduit may be a predetermined temperature.

The end of the breathing conduit temperature is within about 2 degrees Celsius to about 5 degrees Celsius, or about 2.5 degrees Celsius of the desired temperature at the end of the conduit, and optionally, about 2 degrees Celsius to about 5 degrees Celsius, or about 2.5 degrees Celsius less than the predetermined temperature or the therapy parameter.

The end of breathing conduit temperature may be greater than about 25 degrees Celsius, or about 25 degrees Celsius to about 28 degrees Celsius.

The warm up mode comprises controlling a heater of the humidifier to a predetermined temperature, to a predetermined duty cycle, or to a predetermined voltage, or to a predetermined current, or to a predetermined power.

In some configurations the rate of the increase of the temperature (for example to a therapy end of breathing conduit temperature) may be controlled.

The warm up mode may comprise deactivating the flow generator. Alternatively, the warm up mode comprises running the flow generator at a predetermined flow rate or a predetermined flow generator output. In some embodiments, the predetermined flow rate is lower than a therapeutic flow being provided to the patient. In some embodiments, the predetermined flow generator output is a motor speed about 1000 RPM to about 3000 RPM or less than about 2000 RPM.

In some configurations, in the warm up mode may comprise increasing the flow generator output over a predetermined time. The increase may be to a therapy parameter. In some configurations, the flow generator output may be increased from a first level to a second level. For example, in the warm up mode the apparatus may increase the flow rate provided by the flow generator from zero flow rate (or a low flow rate) to a therapy flow rate over a period of time. The predetermined time may be the length of the warm up mode.

In some configurations the rate of the increase of the flow rate, for example to a therapy flow rate, may be controlled.

The end condition of the warm up mode may be when the apparatus reaches one or more therapy parameters (or within a specific margin of the one or more therapy parameters) and/or other desired temperature, and/or after a predetermined. elapsed time.

If the user attempts to start a therapy session before completion of the warm up mode, the apparatus may display a message to the user that the warm up mode has not completed and/or prevent the apparatus from operating in a therapy mode without further confirmation.

As discussed above, the non-therapy mode may be a disinfection mode. The apparatus may operate in the disinfection mode to disinfect the apparatus.

The disinfection mode may be based on the method of disinfecting a breathing assistance apparatus as disclosed in PCT publication no. WO 2007/069922 herein incorporated by reference.

The disinfection mode may allow for a portion of the gases flow path (for example elbow 325, 235) to be disinfected. This may allow safe reuse of the apparatus without having to replace components of the apparatus between uses and between different patients. Disinfection may also allow prevent cross contamination when the apparatus is used with multiple patents and allow safe reuse of the apparatus. Disinfection mode may be utilized in both a home setting and a hospital setting.

When the apparatus is operating in the disinfection mode, the apparatus may be connected to a disinfection conduit. The disinfection conduit may comprise a heater to heat the gases passing through the disinfection conduit.

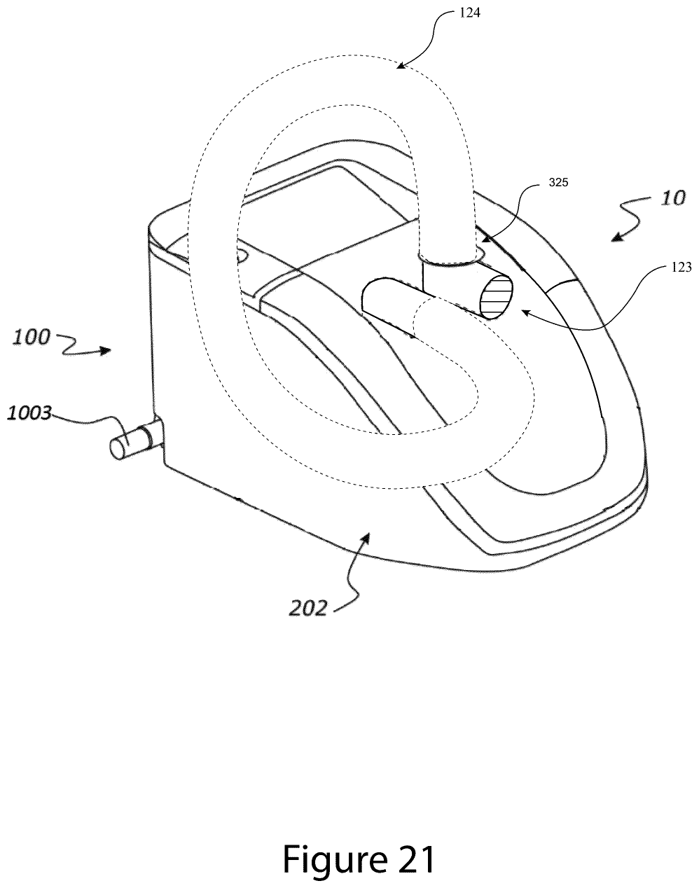

During the disinfection mode, the disinfection conduit 124 may be connected as shown in FIG. 21 to a gases outlet 21 of the apparatus and a flow generator outlet (or a humidification chamber inlet) so that the gas flows from the flow generator through the disinfection conduit, and through the elbow 325 and optionally a filter 123 or valve attached to the elbow 325, to atmosphere.

In the disinfection mode, a heater of the disinfection conduit 124 may be controlled so that the flow of gases in the disinfection conduit 124 reach a predetermined temperature. The predetermined temperature may be about 50 degrees Celsius to about 100 degrees Celsius, or about 60 degrees Celsius to about 90 degrees Celsius.

The predetermined temperature may be a temperature measured by another sensor in the system, for example, a sensor in the elbow 325 or the sensor module 400. Additionally, or alternatively, the predetermined temperature may be a temperature measured by a temperature sensor of the disinfection conduit.

The disinfection mode may comprise controlling the flow generator to provide a predetermined motor speed. The motor speed may be about 1000 RPM to about 6000 RPM, or about 2000 RPM to about 5000 RPM, or about preferably 2000 RPM to about 3000 RPM. In some embodiments, the predetermined motor speed is 2000 RPM.

Alternatively, the disinfection mode may comprise controlling the flow generator to provide a predetermined flow rate, wherein the predetermined flow rate is about 10 litres/minute to about 20 litres/minute.

The apparatus may operate in a disinfection mode when the disinfection conduit is detected to be connected to the apparatus.

Detection of the disinfection tube may be by identifying a heater wire resistance (or for example a heater wire resistance range) specific to the disinfection conduit and/or by an identifier in the conduit e.g. a thermistor or resistor or RFID tag or other identification element.

As discussed above, the non-therapy mode may be a standby mode.

The standby mode may allow for the apparatus to enter a mode which keeps the apparatus ready to operate in a therapy mode if needed.

The apparatus may operate in the standby mode when the user enters an input via the user interface. In some embodiments, the apparatus may operate in the standby mode after detecting that the patient interface has been removed from the user. In some embodiments, the end of therapy command may be generated by detection that the patient interface has been removed from the user for a predetermined amount of time (this predetermined amount of time may be less than the predetermined time required for the apparatus to operate in drying mode, as described in more detail above.)

Detection that the patient interface has been removed may be made as described in more detail above.

The standby mode may comprise running the flow generator at a predetermined flow rate, or a predetermined motor speed.

The predetermined flow rate is lower than a therapy flow rate.

The predetermined motor speed is about 1000 RPM to about 3000 RPM or less than about 2000 RPM.

The apparatus may operate in drying mode after a predetermined time has elapsed while the apparatus has been operating in a standby mode.

The apparatus is configured to collect and store data. The apparatus may collect data at any time. In some embodiments, the apparatus may be configured to collect data while the apparatus is operating in a therapy mode, or a non-therapy mode.

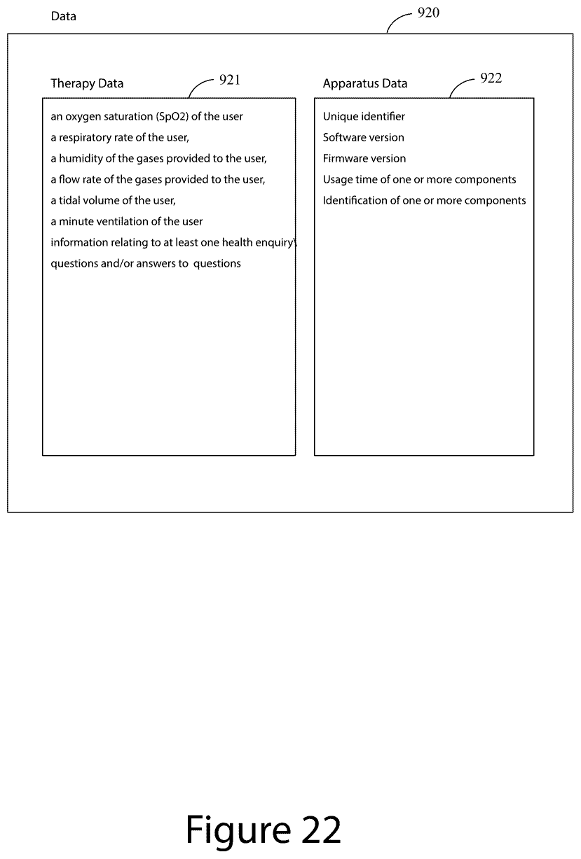

As shown in FIG. 22, the data 920 may comprise therapy data 921 collected during operation in at least one therapy mode. The data may additionally or alternatively comprise apparatus data.

The therapy data 921 comprises data relating to the user and/or therapy provided to the user.

The therapy data 921 may comprise data relating to one or more separate therapy sessions.

In some embodiments, the therapy data comprises data from at least historic therapy mode.

The therapy data 921 may comprise:
a) an oxygen saturation (SpO2) of the user,
b) a respiratory rate of the user,
c) a humidity (dew point) of the gases provided to the user,
d) a flow rate of the gases provided to the user,
e) a patient end temperature,
f) a tidal volume of the user,
g) a minute ventilation of the user,
h) usage data of the apparatus,
i) answers to one of more questions provided to a user,
j) a therapy report (optionally the therapy report relates to previous therapy session report and/or a current finished therapy session),
k) information relating to at least one health enquiry, as described in more detail above,
I) questions of at least one health enquiry,
m) answers of at least one health enquiry,
n) one or more patient baselines,
o) Any combination of a)-n).

In some embodiments, in the non-therapy mode the user is presented with one or more questions and provides answers to these questions via at least one user interface, and wherein the questions and/or answers to the questions form part of the therapy data. It will be appreciated that the questions and answers may relate to a health enquiry or may be separate from a health enquiry.

The therapy data may be collected by one or more sensors as described in more detail elsewhere in the specification.

The apparatus may receive a sensor output from the one or more sensors, and the therapy data is based on the sensor output from one or more sensors.

The one or more sensors may be located within a housing of the apparatus for example Operation sensors 3a, 3b, 3c as shown in FIG. 1. Further sensor locations are shown in FIG. 8A as described below.

The one or more sensors may be located remotely to the apparatus (i.e., outside of the housing) for example patient sensor 26, as shown in FIG. 1.

As shown in FIG. 22, the apparatus data may comprise data relating to one or more properties of the apparatus, and/or the surrounding environment of the apparatus.

The apparatus data may comprise a unique identifier of the apparatus.

The apparatus data may comprise a sensor calibration profile (containing information relating to the calibration of one or more sensors of the apparatus).

The apparatus data may comprise a software and/or firmware version of the software or firmware of the apparatus.

The apparatus data may comprise a total usage time of the apparatus, or a usage time of one or more components of the apparatus (for example a conduit, or a blower).

The apparatus data may comprise identification of one or more components of the apparatus (for example a hardware version of a flow generator, or sensor).

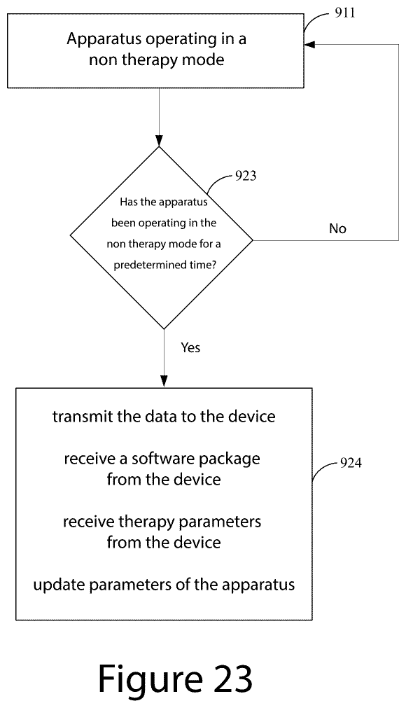

As illustrated in FIG. 23, after a predetermined time operating in the at least one non-therapy mode, the apparatus is configured to undertake one of the following actions 924:
a) transmit the data to a device, or
b) receive a software package from a or the device, or
c) receive therapy parameters (optionally as a prescription) from a or the device, or
d) update parameters of the apparatus, or
e) any combination of a)-d).

The actions 924 may be undertaken concurrently or sequentially.

A number of actions 924 are disclosed, and it will be appreciated that in addition to any combination of actions 924 in any order, any single action can be independently undertaken.

The actions 924 may be performed in a priority order. Each action may have an associated priority and the apparatus may perform the actions in order from the highest priority action to the lowest priority action.

Transmitting the data to a device may be the highest priority task, followed by updating parameters of the apparatus (for example, undertaking sensor calibrations), followed by receiving a software package from a or the device, followed by receiving therapy parameters (optionally as a prescription) from a or the device.

Prioritization of actions may be important when, for example, the apparatus is operating from a battery power source, as there is a chance that the apparatus may shut off it the battery runs out of charge. The actions being undertaken in priority order increases the chance that they will be completed, while the device is still being powered.

In some configurations, when the apparatus is operating from a battery power source the apparatus may prioritize providing power to the components necessary to undertake the actions (for example, the communications module when transmitting or receiving, or the sensors and associated circuitry when undertaking sensor calibration. In some configurations, the actions may be undertaken before other processes as part of the non therapy mode.

In drying mode and disinfection mode, where the flow generator and heater wire are activated, the power draw is increased and therefore priority in power delivery may be given to the components necessary to undertake the actions and/or the actions may be undertaken before other processes as part of the non therapy mode.

In some configurations, the apparatus is configured to: update parameters of the apparatus and then transmit the data to a device. This approach means that sensor calibration information is included in the transmission of the data. This may enable faster alerting of faults (for example via a device such as a phone) and/or recording of faults in a device and/or in the patient and apparatus management (for example to notify a service technician).

In some configurations, the apparatus is configured to: transmit the data to a device, and then receive a software package from a or the device and then update parameters of the apparatus.

In some configurations, the apparatus is configured to: update parameters of the apparatus, and then transmit the data to a device, and then receive a software package from a or the device, and then update parameters of the apparatus based on the software package received from a or the device.

The apparatus may be configured to receive therapy parameters from a or the device after receiving the software package from a or the device. Updating therapy parameters after receiving the software package ensures apparatus software integrity before applying new prescription settings.

The apparatus may be configured to receive therapy parameters from a or the device after transmitting the data to a device This means that updated therapy parameters (for example as a prescription update) can be applied during a non-therapy mode, based on data transmitted from the apparatus. Based on the data transmitted from the apparatus to the device, the apparatus and/or a clinician with access to the patient and apparatus management could make a decision about prescription updates. Further, as some of the non-therapy modes last a predetermined amount of time, there is sufficient time for a clinician to review and approve therapy prescription changes and push them to the apparatus.

The apparatus may be configured to receive therapy parameters from a or the device before updating parameters of the apparatus.

The apparatus may be configured to transmit the data to the device before the apparatus receives therapy parameters from a or the device.

The software package may be an updated software package (compared to the current software package of the apparatus).

In some embodiments, the apparatus is configured to query the device as to whether any updated software packages are available based on a current software package version of the device. Optionally, the apparatus communicates the current software package version, or the timestamp of the current software package to the device.

The apparatus may check the software package version and update the software package on the apparatus if it is an older version.

The apparatus may transmit to the device a confirmation message once the software package is installed successfully and/or transmits an error report if the software package is not installed successfully.

The therapy parameters may be updated therapy parameters (compared to the current therapy parameters of the apparatus).

In some embodiments, the apparatus is configured to query the device as to whether updated therapy parameters are available based on the current therapy parameters of the apparatus. Optionally, the apparatus communicates the current therapy parameter and/or the current therapy parameter version, and/or the timestamp of the current therapy parameters to the device.

The apparatus may be configured to request a software package and/or therapy parameters from the device. The device may then allow the apparatus to initiate receiving of the software package and/or therapy parameters or may provide a location where the apparatus can receive the software package and/or therapy parameters (for example another device).

It will be appreciated receiving a software package and receiving therapy parameters may be initiated by a fetch operation, i.e., the apparatus requests the information from the device, as opposed to a push operation where the device pushes a notification to the device that there is an updated software package and therapy parameters. Alternatively, receiving a software package and receiving therapy parameters may be initiated by a push operation from the device.

FIG. 23 illustrates that when the apparatus is operating in a non-therapy mode, the apparatus conducts a check to determine whether the apparatus has been operating in the non-therapy mode for a predetermined time 923. If the apparatus has been operating in the non-therapy mode for a predetermined time 923 then the apparatus conducts one of the actions in 924 (as set out above).

The device may be:
a) a server (being a local or remote server)
b) a local device
c) a remote device
d) any combination of a)-c)

In some configurations, the device may be, for example, a phone (for example, a smartphone) a computer (for example, a desktop or laptop) and/or a, tablet, and/or a wearable device e.g. a smart watch that has computing and wireless communication capabilities.

The device may be part of a content delivery network or other distributed platform of devices (for example a cloud computing platform).

The apparatus and device may be part of a patient and apparatus management platform.

The patient and apparatus management platform. may be a single server or a network of servers or a cloud computing system or other suitable architecture for operating a patient and apparatus management platform. The patient and apparatus management platform (i.e. including at least one remote server as a device) further includes memory for storing received data and various software applications or services that are executed to perform multiple functions. Then, for example, the patient and apparatus management platform may communicate information or instructions to the apparatus 10 at least in part dependent on the data received. For example, the nature of the data received may trigger the remote server (or a software application running on the remote server) to communicate an alert, alarm, or notification to the apparatus 10.

The patient and apparatus management platform may further store the received data for access by an authorized party such as a clinician or the patient or another authorized party. The patient and apparatus management platform may further be configured to generate reports in response to a request from an authorized party. The reports may comprise responses to health enquiries and/or other patient breathing parameters e.g., respiratory rate or SpO2 and/or parameters e.g., flow rate, humidity level (for example as a therapy report as described above).

Different types of data may be provided to different devices of made available to different parties. For example, apparatus data may be provided to service technicians, while therapy data may be provided to a health practitioner).

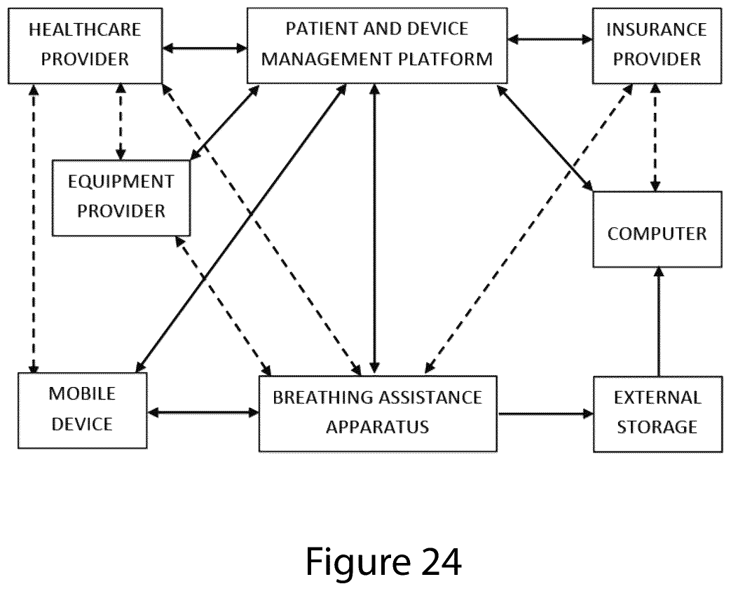

FIG. 24 shows an architecture diagram showing a system for providing breathing assistance or providing respiratory therapy to a patient. The data (answers and/or dashboard and/or plot) may then be provided to an external storage device such as a USB, a patient and apparatus management platform, a mobile device (e.g., smartphone, laptop, tablet, wearable), and an insurance provider or equipment provider. If the data is provided to a USB, then that data may later be downloaded to a computer that can then feed the data to the patient and apparatus management platform or insurance provider. In some embodiments, the mobile apparatus or patient and apparatus management platform may be able to provide data back to the breathing assistance apparatus (e.g., information to the patient about a change in a physiological condition or a pathology of that patient).

It will be appreciated that the device for each of the above may be the same or a different device. For example, the apparatus 10 may transmit the data to the same or a different device as compared to the device the software package is received from.

It will be appreciated that the device could be part of a or patient and apparatus management platform, or the device could be any device, or combination of devices of the a component of the patient and apparatus management platform.

In some embodiments, the action 924 is only undertaken when the apparatus transitions from a therapy mode to a non-therapy mode.

In some embodiments, the action 924 is only undertaken in a non-therapy mode after the therapy session is complete.

The therapy data transmitted to the device may comprise data from at least one therapy mode which has not previously been transmitted to the device.

The data may be transmitted to the device even if the apparatus has not operated in at least one therapy mode.

The data may be transmitted to the device after a predetermined period of time has elapsed since a last transmission of data to the device (optionally the predetermined period of time is 24 hours).

The predetermined time may be about 5 minutes to about 25 minutes.

In some embodiments, the predetermined time is greater than 5 minutes.

In some embodiments, the predetermined time is less than a total length of time the apparatus is configured to operate in the non-therapy mode. For example, if the drying mode is configured to operate for a predetermined amount of time (for example as a drying process)—i.e., 90 minutes, then the predetermined time would be less than 90 minutes.

In some embodiments, the predetermined time is a proportion of a total length of time the apparatus is configured to operate in the non-therapy mode. In some embodiments, the proportion of a total length of time the apparatus is configured to operate in the non-therapy mode is less than about 50%, or less than 40%, or less than about 30%. For example, if the drying mode is configured to operate for a predetermined amount of time (for example as a drying process)—i.e., 90 minutes, then the predetermined time would be, at a proportion of, for example, 50%-45 minutes.

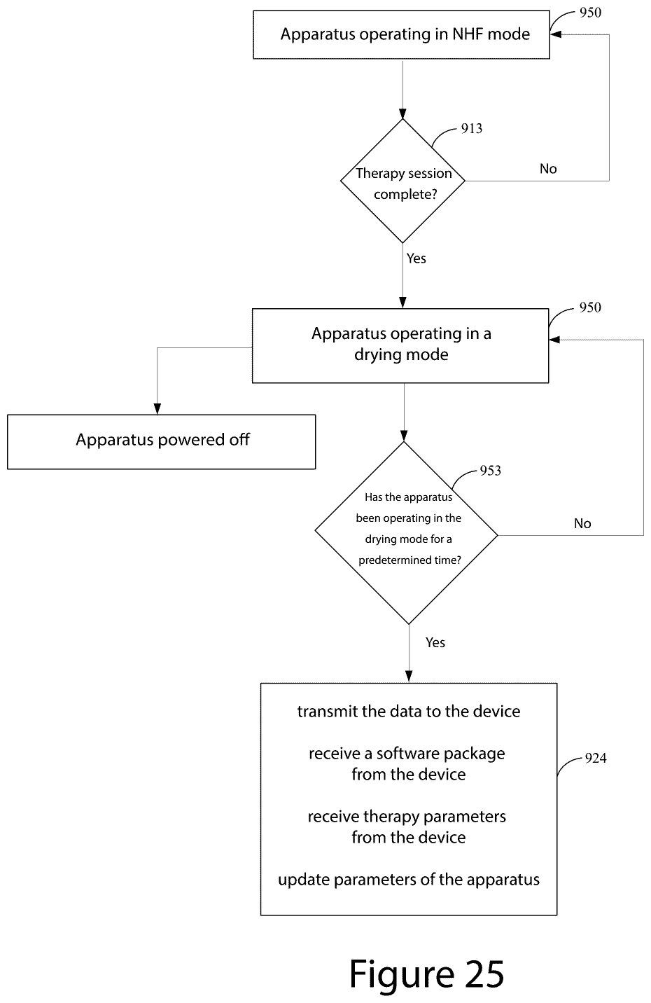

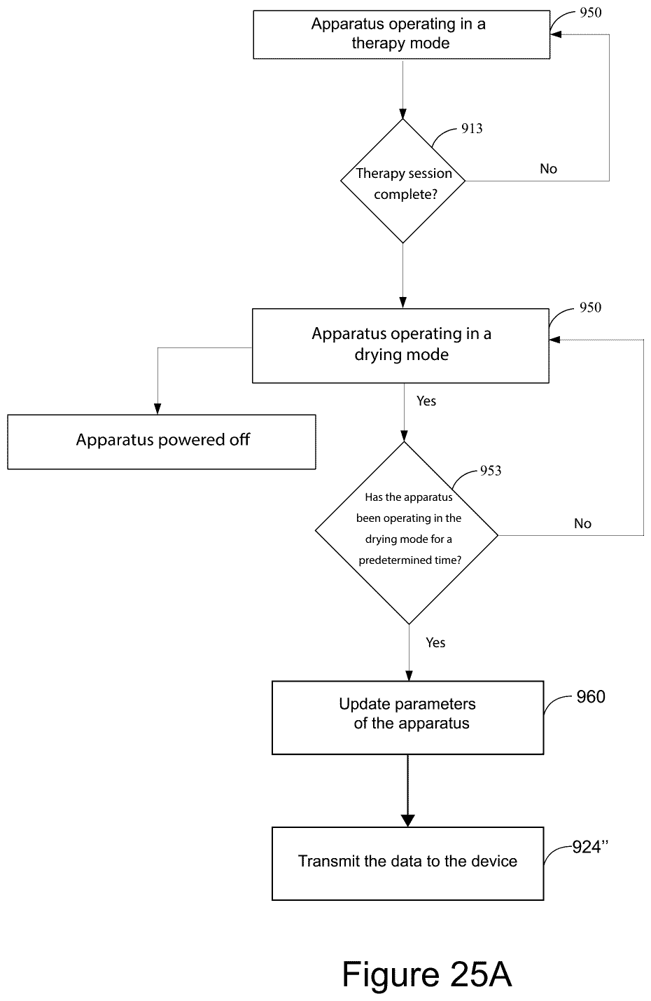

By way of example, as shown in FIG. 25, the apparatus may, after operating in a nasal high flow (NHF) mode 950, and that the therapy session is complete 913 (for example by the user indicating that the therapy session has completed), operate in the drying mode. In the drying mode, the heater of the humidifier is deactivated, and a heater of the conduit is activated while the flow generator provides gases at a predetermined flow rate. While operating in the drying mode, the user may power off the apparatus before the drying mode has completed. If the user attempts to turn the apparatus off before the drying mode has completed the apparatus may display a message warning that user that the drying mode is incomplete.

After the apparatus has been operating in the drying mode for a predetermined time 953 (in this case 10 minutes), the apparatus may undertake one or more actions 924. For example, the apparatus may transmit data (including therapy data and apparatus data) to a device (for example a server), receive a software package from a device (for example, from the same or a different server), receive therapy parameters package from a device (for example, from the same or a different server).

While the apparatus undertakes the action 924, the apparatus may display a message warning the user not to power off the apparatus.

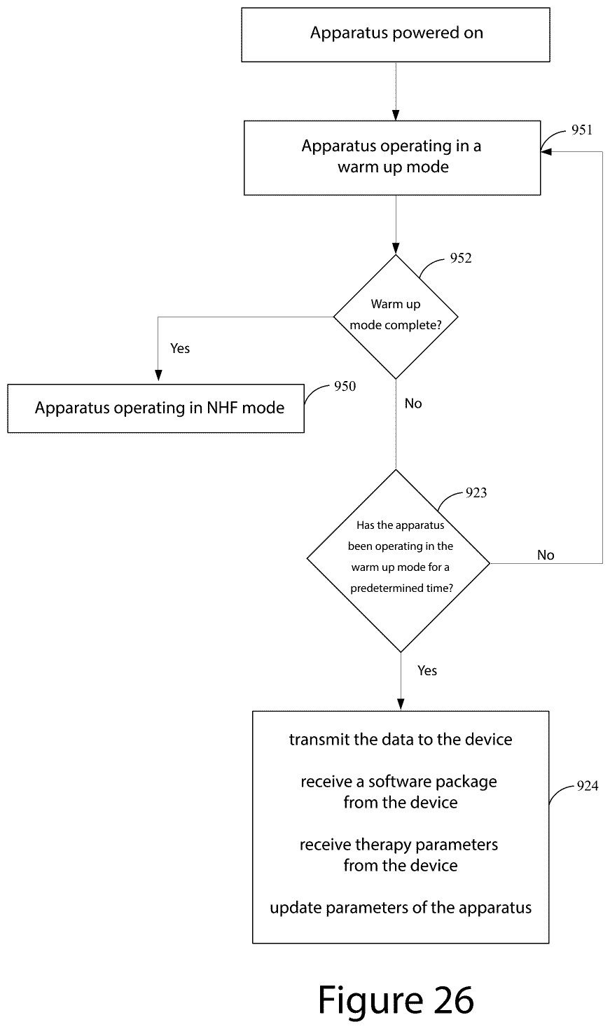

By way of example, as shown in FIG. 26, the apparatus may, after powering on, operate in the warm up mode 951. In the warm up mode, the heater of the humidifier and the heater of the breathing conduit are activated while the flow generator is deactivated.

The apparatus 10 may operate in a NHF mode 950 if the warm up mode is complete (for example if the user indicates the start of a therapy session). If the warm up mode has not reached its end condition (for example, a desired temperature and/or a predetermined elapsed time) the apparatus may display a message to the user that the warm up mode has not completed.

After the apparatus has been operating in the warm up mode for a predetermined time (in this case, 10 minutes), the apparatus may undertake one or more actions 924. For example, the apparatus may transmit data (including therapy data and apparatus data) to a device (for example a server, receive a software package from a device (for example, from the same or a different server), receive therapy parameters package from a device (for example, from the same or a different server).

The user may be presented with a visual indicator and/or an audio indicator if the user attempts to power off the apparatus which the data is being transmitted to the device.

In some embodiments, the user is presented with a visual indicator and/or an audio indicator while the data is being uploaded to the device.

The visual indicator may be presented on the display module.

The visual indicator may be for example a picture of a modem icon, and/or a message.

The apparatus may be prevented from powering off while the action is being undertaken. However, the potential inconvenience for the user in this case is limited, as it is unlikely they will want to power off the apparatus before the end of the non-therapy process.

In a non-therapy mode, the apparatus may display a visual indicator on the display to warn a user not to use the apparatus during the non-therapy mode.

The apparatus operating for a predetermined time in a non-therapy mode ensures that the action 924 is not undertaken if the apparatus is powered off immediately after the apparatus exits the therapy mode, as this could jeopardize the completion of the action 924.

To facilitate the transmission of data, and/or to receive the software package and/or to receive the therapy parameters from the apparatus, the apparatus is configured to activate a network interface (as described in more detail above) and establish a connection with the device to transmit data to the device, and/or receive a software package and/or therapy parameters from a or the device.

The software package may comprise a firmware update (for example to control one or more hardware functions of the apparatus). Additionally, or alternatively, the software package may comprise a software update (for example to update algorithms or routines in the apparatus, for example, a control algorithm).

In some embodiments, after receiving the software package, the apparatus applies the software package to the apparatus.

The ability to receive updates in this way ensures timely roll-out of updates which may improve apparatus performance.

The predetermined time delay after entering a non-therapy mode may be important for applying a software package to the apparatus, as if the apparatus is powered off during installation of the software package this may cause the software package not to be applied, or to be partially applied which may affect functionality of the apparatus. For example, if the software package is applied as soon as the therapy session has ended, the user may power off the apparatus after therapy. Further, if the software package is only applied upon selection by the user (to mitigate risk that the apparatus is powered off) the user may not apply important software packages due to inconvenience. In another example, if the software package is applied during therapy there is a risk that inadequate therapy is provided for a period of time.

Updating a parameter of the apparatus may comprise updating a calibration parameter of the apparatus. Updating a parameter of the apparatus may comprise undertaking a sensor recalibration (for example, as described in more detail below).

The apparatus may receive one or more updated parameters for the apparatus from a device and apply these updated parameters to the apparatus. The updated parameters for the apparatus may for example include the predetermined time after operating in a non-therapy mode when the action is undertaken, or sensor calibration settings. The updated parameters may be received as part of the software package.

In some embodiments, after receiving the therapy parameters from a or the device, the apparatus applies updates the therapy parameters of the apparatus (for example as a prescription update). The therapy parameters may be specific to a particular therapy mode. The update to the therapy parameters may also only update a subset of the therapy parameters of the apparatus, and not necessarily all the therapy parameters of the apparatus.

For example, the update to the therapy parameters may comprise a change in, for example, an oxygen concentration of the gases provided to the user, and/or a change in the humidity level provided to a user.

In addition, because the action 924 is only undertaken in the non-therapy mode, there is a decrease in the transmission and receiving of data, meaning that data costs can be reduced as compared to where the apparatus is constantly transmitting and receiving data.

The apparatus 10 may transmit a signal to the device every predetermined amount of time (for example at predetermined time intervals) if the apparatus has not been operated in a therapy mode. In some embodiments, the predetermined amount of time is 24 hours. In this case, the apparatus data may also be transferred at the same time.

If the transmission of this signal is unsuccessful (e.g., due to a connection fault), the apparatus will re-attempt transmission after a certain time period, and up to a certain number of attempts until 24 hours elapses. If unsuccessful after said certain number of attempts, the apparatus will wait until for another 24-hour window and then restart the process.

The device may send a notification to the apparatus that an update in therapy parameters, or a software package is available. In this case the apparatus may download the update from the device, but wait until the predetermined time has elapsed in the non-therapy mode, before applying the update.

When the apparatus is operating via a battery for example with the battery as a power source (as, for example, as described above) the apparatus may, after the predetermined time in a non-therapy mode, not undertake the action, or may prompt the user to confirm the action before undertaking the action. This may preserve the charge of the battery or allow for the user to have more control over the power usage of the apparatus.

In some embodiments, when the apparatus is operating via a battery power source, a user may be able to manually prompt the apparatus to undertake the action (optionally via an input on the user interface).

In some embodiments, when the apparatus is operating via a battery power source, a user may be able to manually prompt the apparatus to undertake the action after the predetermined time operating in the non-therapy mode.

In some embodiments, when the apparatus is reconnected to an external power source the apparatus may prompt the user to perform the action (for example if there is data to transmit, or it is time for the apparatus to check for an updated software package.)

In some embodiments, when the apparatus is reconnected to an external power source the apparatus may prompt the user to perform the action after the predetermined time operating in the non-therapy mode.

When the apparatus is entered into a battery charging state (for example, when connected to an external power source), the apparatus may be configured to immediately perform the action or perform the action after a predetermined time in the battery charging state (optionally after the predetermined time operating in the non-therapy mode).

The predetermined time may be the predetermined time as described above in relation to operating in a non-therapy mode, or more than about 2 minutes, or more than about 5 minutes.

In some embodiments, the battery charging state may be entered into when the apparatus is not powered on.

When a service task is completed (for example, general servicing of the apparatus, or a replacement of a component of the apparatus), the apparatus may be configured to immediately perform the action or perform the action a predetermined time after the service task is complete.

In some embodiments, after the service task is complete the apparatus may prompt the user (in this case a service technician) to undertake the action (as described above).

In the context of the action being performed after a service task, the action may act as a connectivity test to ensure that the communications module is operational.

When the apparatus is entered into a travel state (for example for air travel or travel away from a user's normal location) the apparatus may, after the predetermined time in a non-therapy mode, not undertake the action, or may prompt the user to confirm the action before undertaking the action. This may limit data transfer costs, or converse battery charge.

In some embodiments, when the apparatus exits the travel state the apparatus may prompt the user to perform the action (for example, if there is data to transmit, or it is time for the apparatus to check for an updated software package.)

When the apparatus is in a travel state a user may be able to manually prompt the apparatus to undertake the action (optionally via an input on the user interface).

In a travel state, the apparatus may deactivate the communications module (or part of the communications module) in accordance with regulations relating to aircraft travel. In some embodiments, in a travel state the apparatus may also operate via a battery.

As described above, the apparatus is configured to operate in at least one therapy mode and at least one non-therapy mode.

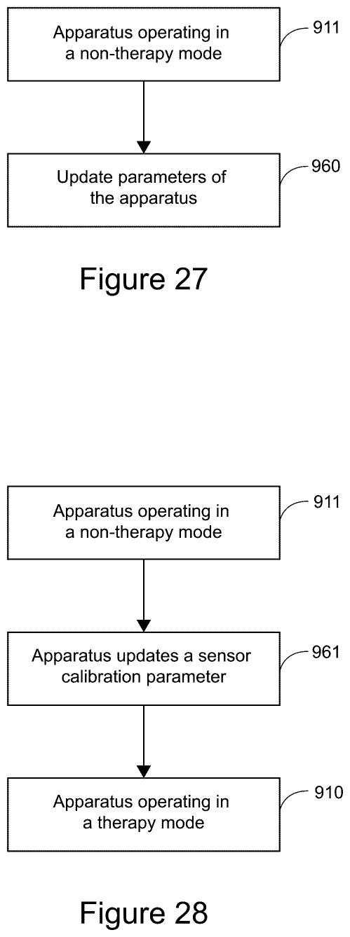

As shown in FIG. 27, when the apparatus is operating in the at least one non-therapy mode 911, the apparatus is configured to update parameters of the apparatus 960. The non-therapy mode may provide a safe opportunity (for example as described in more detail above) for the apparatus to update parameters of the apparatus. The updated parameters may be used during a therapy mode 910—for example, sensor calibration parameters 961 updated in a non-therapy mode 911 may be used in the therapy mode 910 (as, for example, shown in FIG. 28).

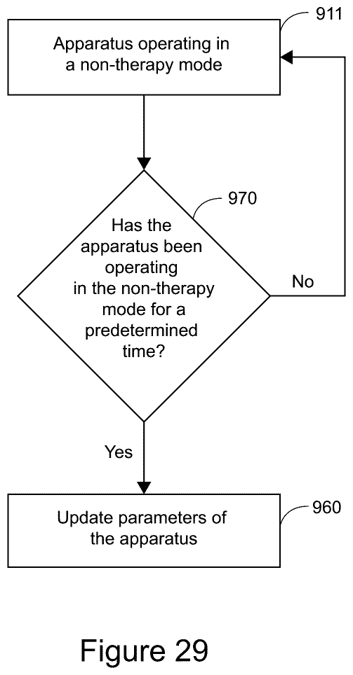

As shown in FIG. 29, the apparatus is configured to update parameters of the apparatus 960 after a predetermined time of the apparatus operating in the at least one non-therapy mode 970.

In some configurations, the apparatus may be configured to update parameters of the apparatus 960 after about 10 minutes of the apparatus operating in the non-therapy mode.

In some configurations, the apparatus may be configured to update parameters of the apparatus 960 about 5 minutes before the end of the apparatus operating in the non-therapy mode.

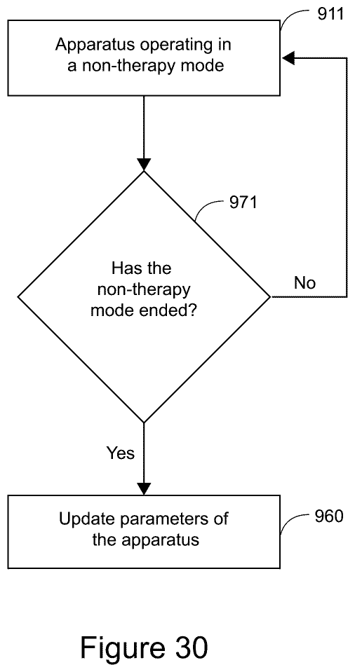

Alternatively, or additionally, as shown in FIG. 30, the apparatus is configured to update parameters of the apparatus 960 at the end of a non-therapy mode 971.

In some configurations, for example, as part of step 924 in FIG. 25, the apparatus may be configured to update parameters of the apparatus before transmitting data to the device. The data transmitted may, for example, include the updated parameters of the apparatus.

As shown in FIG. 25A, after operating in a therapy mode 950, for example, as the therapy session is complete 913 (for example, after an end of therapy command as described in more detail above), the apparatus may operate in the drying mode. In the drying mode, the heater of the humidifier is deactivated, and a heater of the conduit is activated while the flow generator provides gases at a predetermined flow rate. While operating in the drying mode, the user may power off the apparatus before the drying mode has completed. If the user attempts to turn the apparatus off before the drying mode has completed, the apparatus may display a message warning the user that the drying mode is incomplete.

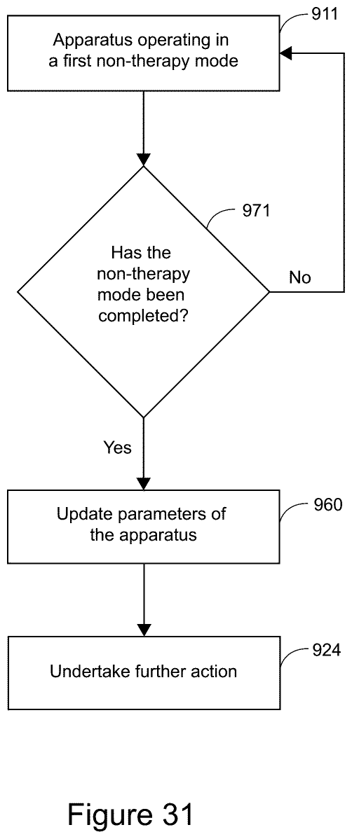

After the apparatus has been operating in the drying mode for a predetermined time 953 (in this case 10 minutes), the apparatus may update at least one parameter of the apparatus 960. The parameter may comprise one or more sensor calibration parameters as described in more detail elsewhere. After the one or more sensor calibration parameters have been updated, the apparatus may proceed to step 924″ where the apparatus transmits data to the device. The data may comprise the one or more sensor calibration parameters. The data may comprise other data as described in more detail above, for example, therapy data and/or apparatus data. In some configurations, the apparatus may undertake one or more action 924 after the one or more sensor calibration parameters have been updated (as, for example, described in more detail elsewhere and, for example, as shown in FIG. 31).

While the apparatus undertakes the action 924, the apparatus may display a message warning the user not to power off the apparatus.

As shown in FIG. 31, the apparatus may be configured to update parameters of the apparatus 960 at the end of a non-therapy mode 972 and subsequently undertake a further action as described in more detail above. In some configurations, the apparatus may provide a prompt to the user via a user interface to confirm updating parameters of the apparatus.

Updating the parameters of the apparatus during some non-therapy modes (for example, drying mode and/or warm up mode and/or cool down mode and/or disinfection mode) may allow for the apparatus to efficiently use the time the apparatus is in the non-therapy modes. This means that parameters of the apparatus may be updated while the apparatus is undertaking some non-therapy modes which happen in normal operation of the apparatus. This may mean that the apparatus does not need to stop undertaking some non-therapy modes to update parameters of the apparatus.

Further, as described above, the apparatus does not need to update parameters of the apparatus during a therapy mode. Updating parameters of the apparatus during a therapy mode may increase patient risk, as updating may mean that therapy needs to stop being delivered or not provided to the required therapy parameters.

Having a separate mode where the apparatus updates parameters may be less convenient for the user, as it has to be positively selected by a user, and this may discourage a user from updating the apparatus. This may lead to an apparatus which is not frequently updated, which may compromise therapy. Preventing the apparatus from operating when the parameters have not been updated may be a risk, for example, when the user requires therapy but must wait for the apparatus to update parameters before use.

In some configurations, the updated parameters of the apparatus may comprise sensor calibration parameters of at least one sensor.

Sensor accuracy may degrade over time or be compromised by damage or incorrect use. If the sensors are out of calibration the apparatus may not be able to provide adequate therapy. If sensors are out of calibration then the control systems of the apparatus may be unable to control functions of the apparatus.

For example, if the flow rate sensor (i.e. an ultrasonic flow rate sensor) is out of calibration then the apparatus may not be able to accurately provide flow based therapies (for example high flow therapy).

By way of further example, if the pressure sensor is out of calibration, then the apparatus may not be able to accurately provide pressure based therapies (for example CPAP, BCPAP and/or Bilevel therapies).

By way of further example, if the oxygen concentration sensor (i.e. an ultrasonic oxygen concentration sensor) is out of calibration, then the apparatus may not be able to accurately control the amount of oxygen provided to a user. This may mean that the user isn't provided with the therapy oxygen concentration, and the user could be under or over oxygenated which may lead to negative health outcomes.

Updating sensor calibration parameters may ensure safe and proper functioning of the apparatus.

In some configurations, the at least one sensor may be any sensor in the apparatus. For example, the at least one sensor may be any sensor as described elsewhere in the specification. In some configurations, the at least one sensor may be associated with the apparatus. For example, the at least one sensor may be connected to the apparatus.

The at least one sensor may be any one of:
a pressure sensor,
a temperature sensor,
a humidity sensor,
an oxygen concentration sensor,
a flow rate sensor.

The pressure sensor may be an absolute pressure sensor or a differential pressure sensor.

The at least one sensor may be part of the sensor module (as described in more detail above). In some configurations, a sensor assembly may comprise at least one, or a plurality of sensors (for example, a temperature and humidity sensor).

The sensor may be configured to measure a characteristic (for example, a characteristic of the gases), for example, the pressure sensor would be configured to measure pressure of the gases.

As described in more detail above, with respect to, for example, FIG. 8A, the at least one sensor may be located in one or more of:
a sensor module (optionally the sensor module may be located as part of the flow generator and/or between the blower and the humidifier)—for example, sensor 44,
the flow generator—for example, sensor 3a, 44 and/or 43,
a location upstream of the flow generator—for example, sensor 41 and/or 42,
a location downstream of the flow generator—for example, sensor 44, 46 and/or 48,
the humidifier—for example, sensor 45 and/or 46,
a location upstream of the humidifier—for example, sensor 41, 42 and/or 44,
a location downstream of the humidifier—for example, sensor, 46 and/or 48,
a conduit configured to be connected to a gases outlet of the humidifier and convey the flow of gases to the user (optionally at a user end of the conduit near a patient interface)—for example, sensor 48
a patient interface,
an ambient sensor—for example, sensor 40, a measuring chamber (optionally as part of the sensor module)—for example, sensor 44,
a humidification inlet and/or a humification chamber inlet,
a humidification outlet and/or a humification chamber outlet—for example, sensor 46,
a control board—for example, sensor 43, 44, and/or 47.

By way of example, operation sensors, 3a, 3b, 3c, and 29 are included in the apparatus 1 as shown in FIG. 1, and/or sensors 40, 41, 42, 43, 44, 45, 46, 47 and 48 as shown in FIG. 8A. The operation sensors may be in any of the locations as described above, or any of the sensors as described above.

The sensor calibration parameters may relate to a relationship between an output of the at least one sensor and the characteristic the sensor is configured to measure. The sensor calibration parameters may comprise one or more of:
a calibration factor,
a calibration curve,
an internal parameter of the sensor.

For example, the calibration factor or curve may be a factor or curve which is used as part of a formula to determine the relationship between the output of the sensor and the characteristic the sensor is configured to measure.

The correction curve or factor may be, for example, an offset applied to the sensor output.

In some configurations, the sensor calibration parameter may be a particular formula used (for example, based on a selection from a plurality of formulae).

In some configurations, internal parameters of the sensor may be updated. The internal parameter may be used by the sensor in providing an output.

The apparatus may be configured to update sensor calibration parameters of the at least one sensor at least once, and optionally, a plurality of times. For example, the apparatus may update sensor calibration parameters of the at least one sensor, and after a predetermined time update sensor calibration parameters of the at least one sensor again. For example, during a non-therapy mode the sensor calibration parameters may be updated a plurality of times.

The apparatus may be configured to update the sensor calibration parameters based on the output of the at least one sensor and a further sensor. The output of one sensor may be compared to the output of the further sensor, and the sensor calibration parameters determined based on the comparison.

The sensor calibration parameters may be used by the apparatus to determine the characteristic the at least one sensor is configured to measure based on the output of the at least one sensor. Furthermore, the apparatus may be configured to update a control scheme based on the sensor calibration parameters. For example, a different humidity control scheme may be used to control humidity output of the apparatus (for example, the humidifier of the apparatus) based on the sensor calibration parameter.

The sensor calibration parameters may be stored in a memory of the apparatus.

The sensor calibration parameters may be used by the apparatus in a therapy mode.

The apparatus may be configured to update the sensor calibration parameter if an error of the sensor is outside of an allowable tolerance. The allowable tolerance may be based on the specific type and/or location of the sensor. For example, an oxygen concentration sensor (as described in more detail below) may have an allowable tolerance of +/−3%.

The allowable tolerance may comprise an allowable specification tolerance and/or an allowable therapy tolerance. The allowable specification tolerance may be based on the particular specifications of the sensor and being outside the allowable specification tolerance may indicate a faulty sensor (i.e., a sensor calibration failure). The allowable therapy tolerance may be a tolerance associated with the ability of the sensor to be acceptable for use in providing therapy.

The allowable therapy tolerance may be based on the specific sensor location and sensor type. The allowable therapy tolerance may be based on the of the apparatus control system and the use of the sensor in control.

If the allowable specification tolerance is not met, then the apparatus may return a fault and prevent use of the apparatus i.e., as sensor calibration failure.

If the allowable therapy tolerance is not met, then the apparatus may update the sensor calibration parameter for the sensor.

If the error of the sensor is within the allowable tolerance (i.e., a sensor calibration success), then the apparatus may not update the sensor calibration parameter for the sensor and continue using a current sensor calibration parameter. If the error of the sensor is outside the allowable tolerance, then the apparatus may update the sensor calibration parameter for the sensor which may also be classified as a sensor calibration success.

The error of the sensor may be, for example, determined by comparison between outputs of sensors (for example, the first sensor and second sensor as described below) in the apparatus, and/or by undertaking specific tests as described in more detail below.

It will be appreciated, in situations where the error is based on the comparison between outputs of sensors, the one of the sensors is assumed to be correct and the error is determined from this sensor. The particular sensor which is assumed to be correct will be dependent on, for example, sensor type, sensor location, and sensor error and the circumstances of the apparatus. The apparatus may perform one or more checks on the particular sensor which is assumed to be correct before assuming it is correct. For example, if the assumed correct sensor is a temperature sensor, and the temperature is outside an acceptable range the apparatus may not assume the sensor to be correct.

If the apparatus detects that the sensor is out of the allowable tolerance and/or if the sensor calibration parameter changes by more than a threshold then the apparatus may undertake any combination of the following:
a) Raise an alarm on the apparatus (for example, audio and/or visual alarm)
b) Transmit an alarm condition to a device (for example, a device as described elsewhere in the specification, the device may for example be a user device or a clinician device)
c) Prevent operation of the apparatus (in some configurations after the user acknowledges the alarm the apparatus may be allowed to be operated, in some configurations a service technician may need to repair and/or service the apparatus before the apparatus is allowed to operate.)
d) Generate a report, the report containing information relating to the sensor calibration process (for example, including the error and whether the sensor is within tolerance or outside of tolerance, the success or failure of the calibration and/or a resolution to the fault of the sensor). The report may be transmitted to the device, and optionally be part of the data transmitted to the device. In some configurations the report may be displayed on a display of the apparatus and/or a display of the device.
e) Display via a display of the apparatus an/or a display of the device information relating to the sensor calibration process (for example, including the error and whether the sensor is within tolerance or outside of tolerance, the success or failure of the calibration and/or a resolution to the fault of the sensor).

Described below are a number of examples of updating a sensor calibration parameter for at least one sensor. It will be appreciated that any combination of the below examples may be combined.

Sensor calibration parameters may be updated for a at least one of the sensors. The sensor calibration parameters may be updated either concurrently or sequentially.

Multiple tests can be undertaken for each sensor to update the sensor calibration parameters associated with the sensor. For example, for a flow rate sensor a no flow rate calibration could be undertaken in combination with a predetermined flow rate calibration (as described in more detail below).

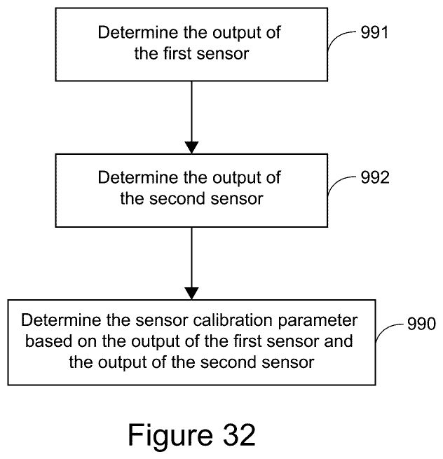

In some configurations, the at least one sensor comprises a first sensor and the further sensor comprises a second sensor. The apparatus 1 may be configured to update the sensor calibration parameters relating to a first sensor based on an output of a second sensor. The sensor calibration parameters may be based on a comparison between an output of the first sensor based on the output of a second sensor. For example, as shown in FIG. 32, at step 991, the apparatus 1 determines the output of the first sensor, at step 992, the apparatus 1 determines the output of the second sensor, and at step 990, the apparatus 1 determines the sensor calibration parameter based on the output of the first sensor and the output of the second sensor. It will be appreciated that steps 991 and 992 can be undertake in parallel.

The first sensor may be an ambient sensor and the second sensor may be a sensor located in a flow path of the apparatus.

In the non-therapy mode, the controller may control the flow generator such that no flow of gases is provided from the flow generator so that the conditions of the gases in the flow path are the same as the ambient conditions.

In some configurations, the first sensor may be located in the same location as the second sensor (for example, as ambient sensors, or located in the flow path of gases).

Specific examples relating to a pressure sensor, a temperature sensor, a flow rate sensor and a humidity sensor are outlined below. It will be appreciated in the examples below that the first sensor and second sensor may be swapped.

In some configurations, the at least one sensor comprises a first pressure sensor and the further sensor comprises a second pressure sensor. The apparatus 1 may be configured to update the sensor calibration parameters relating to a first pressure sensor based on an output of a second pressure sensor. The sensor calibration parameters may be based on a comparison between an output of the first pressure sensor and the output of a second pressure sensor.

The first pressure sensor may be an ambient pressure sensor and the second pressure sensor is a pressure sensor in located a flow path of the apparatus.

In some configurations, the pressure sensors may be the sensor 40, 41, 42, 44, 46 and/or 48.

In some configurations, when the apparatus is calibrating the supplemental gas inlet sensor 42 the apparatus may prompt a user to confirm that no gases supply is connected to the supplemental gas inlet such that the ambient pressure is the same as the pressure at the supplemental gas inlet.

The apparatus may determine whether a gases supply is detected to one or both of the supplemental gases inlet and/or the alternative gases inlet. The method of detecting the gases supply may be that as disclosed in PCT publication no. WO 2021/048744 which is hereby incorporated by reference in its entirety.

The presence of a gases supply connected to the supplemental gas inlet may be detected by pulsing the valve (for example, transiently opening it fully and then closing it) and observing subsequent fluctuations in gas composition (via the oxygen concentration sensor). During this procedure, the blower may be providing a flow rate at a predetermined flow rate.

In the non-therapy mode, the controller may control the flow generator such that no flow of gases is provided from the flow generator so that the ambient pressure is the same as the pressure of the gases in the flow path (i.e., the flow path of the gases is the same pressure as ambient).

In some configurations, the first pressure sensor may be located in the same location as the second pressure sensor (for example, as ambient sensors, or located in the flow path of gases).

In some configurations, during a non-therapy mode, the apparatus may control the flow generator to provide no flow of gases (i.e. controlling the flow generator to a no flow rate). For example, where the non-therapy mode is a drying mode (where the flow generator is controlled to provide a predetermined flow rate and/or motor speed) the apparatus may control the flow generator to a no flow rate (for example, by shutting off the blower) and update at least one sensor calibration parameter based on a comparison between an output of the first pressure sensor based on the output of a second pressure sensor. The apparatus may update the sensor calibration parameter a number of times during the drying mode.

The no flow rate may be zero flow rate or so close to zero such that the flow rate of the gases is insignificant.

In some configurations, the pressure sensor may be a differential pressure sensor configured to measure a pressure difference between the ambient environment and the gases flow path. In this case, the sensor calibration parameter may be determined based on the output of the pressure sensor when no flow of gases is provided (as described above), so that the ambient pressure is the same as the pressure of the gases in the flow path and the output of the differential pressure sensor should read zero.

The allowable tolerance for the pressure sensor may be about +/−2 cmH₂O to about +/−3 cmH₂O, or about +/−5 cmH₂O to about +/−15 cmH₂O, or about +/−10 cmH₂O. The error may be the difference between the output of the first pressure sensor and the second pressure sensor.

In some configurations, the at least one sensor comprises a first temperature sensor and the further sensor comprises a second temperature sensor. The apparatus is configured to update the sensor calibration parameters of a first temperature sensor based on the output of a second temperature sensor. The sensor calibration parameters may be based on a comparison between an output of the first temperature sensor and the output of a second temperature sensor.

The first temperature sensor may be an ambient temperature sensor (for example additional sensors 40), and the second temperature sensor may be a patient end temperature sensor located in or near a patient end of conduit configured to be connected to the apparatus. In some configurations, the first temperature sensor may be a patient end temperature sensor located in or near a patient end of conduit configured to be connected to the apparatus, and the second temperature sensor may be an ambient temperature sensor.

The first temperature sensor may be located at the same location as the second temperature sensor (for example, as ambient sensors, or located in the flow path of gases).

The allowable tolerance for the temperature sensor may be about +/−0° C. to about +/−3° C., or about +/−0.5° C. to about +/−1° C., or about +/−3° C. The error may be the difference between the output of the first temperature sensor and the second temperature sensor.

In some configurations the temperature sensors may be the sensor 40, 41, 42, 44, 46 and/or 48.

In some configurations, the at least one sensor comprises a first flow rate sensor and the further sensor comprises a second flow rate sensor. The apparatus 1 may be configured to update the sensor calibration parameters relating to a first flow rate sensor based on an output of a second flow rate sensor. The sensor calibration parameters may be based on a comparison between an output of the first flow rate sensor and the output of a second flow rate sensor.

The first flow rate sensor may be a hot wire anemometer (for example, a heated thermistor) type and the second flow rate sensor may be an ultrasonic sensor (as described in more detail elsewhere). In some configurations, the first flow rate sensor may be an ultrasonic sensor and the second flow rate sensor may be a hot wire anemometer (for example, a heated thermistor) type sensor.

The allowable tolerance between the first flow rate and the second flow rate sensor may be about +/−0.5 LPM to about +/−5 LPM, or about +/−1 LPM to about +/−4 LPM, or about +/−3 LPM, or about +/−2.5 LPM.

The error may be the difference between the output of the first flow rate sensor and the second flow rate sensor.

In some configurations, the flow rate sensors may be the sensor 44 (and for example be thermistor 2206 and ultrasonic transducers 2204).

In the non-therapy mode, the apparatus may be configured to provide no power to a heater of the humidifier and/or a heater wire of a conduit for a predetermined period of time, so that the ambient temperature is the same as the temperature of the gases in the flow path. This may be undertaken towards the end of the non-therapy mode (for example, a predetermined time before the end of the non-therapy mode) and/or after other sensor calibration parameters have been determined as described elsewhere.

The apparatus may comprise at least one valve. The at least one valve may be, or be part of, the valve module as described in more detail above.

The at least one valve may be configured to be connected to a source of a supplemental gas; the supplemental gas may be for example oxygen.

The at least one valve may control a flow of the supplemental gas. In some configurations, the at least one valve may control the flow of the supplemental gas to reach a supplemental gas concentration. For example, the at least one valve may control the supplemental gas concentration (of the flow of gases provided to the user) to reach a therapy patient oxygen saturation. In some configurations the apparatus may further comprise at least one patient oxygen saturation sensor, and the operation of the at least one valve to control the supplemental gas concentration to reach the therapy patient oxygen saturation may be based on an output of the at least one patient oxygen saturation sensor. By way of further example, the apparatus may be configured to operate the valve to control the supplemental gas concentration so that the flow of gases may be provided to the user at a therapy oxygen concentration.

The flow of supplemental gas may be configured to be combined with ambient air. The combined supplemental gas and ambient air may be provided to the flow generator.

In some configurations, the flow of supplemental gas may be configured to be added to the flow of gases generated by the flow generator.

In some configurations, when the apparatus is updating parameters, the at least one valve may be operated to prevent the flow of supplemental gas: when the at least one valve is operated as such it may be assumed that the concentration of the supplemental gas in the flow of gases is the concentration of the supplemental gas in ambient air. In some configurations, before the apparatus updates parameters of the apparatus, the apparatus may be configured to prompt a user (optionally via a user interface) to disconnect the source of the supplemental gas from the alternative supply inlet.

The alternative supply inlet may be configured to be connected to a source of a supplemental gas. The flow of supplemental gas from the alternative supply inlet may be configured to be combined with ambient air, and the combined supplemental gas and ambient air may be provided to the flow generator. The flow of supplemental gas from the alternative supply inlet may be configured to be added to the flow of gases generated by the flow generator.

The at least one sensor may be an oxygen concentration sensor. The oxygen concentration sensor may for example comprise an ultrasonic sensor. The ultrasonic sensor may be the ultrasonic transducer as described in more detail above.

The apparatus may be configured to determine an output of the oxygen concentration sensor when no supplemental gas is provided as part of the flow of gases and/or when supplemental gases are provided as the part of the flow of gases. The output of the oxygen concentration sensor may be indicative of an oxygen concentration of the flow of gases.

The apparatus may be configured to determine an oxygen concentration sensor calibration parameter.

The apparatus may be configured such that no supplemental gas is provided as part of the flow of gases. Configuring the apparatus 1, such that no supplemental is be provided as part of the flow of gases may be by controlling the at least one valve to provide no supplemental gas, and/or by prompting the user to take an action to prevent supplemental gas from being provided (for example, by disconnecting a supplemental gas source).

In some configurations, (for example, when it is confirmed no supplemental gas is provided as part of the flow of gases) the oxygen concentration sensor calibration parameter is determined based on the output of the oxygen concentration sensor and/or an estimated oxygen ambient concentration. The estimated oxygen ambient concentration may be for example about 19% to about 23%, or about 20.9%, or about 21%, or about 22%.

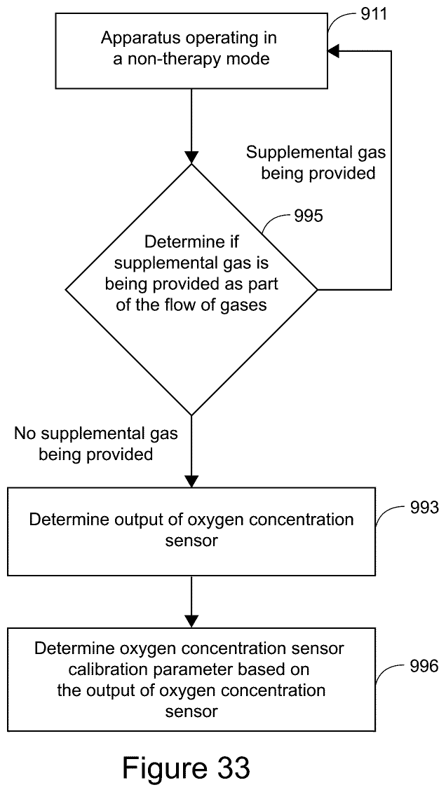

For example, as shown in FIG. 33, at step 911 the apparatus is operating in a non-therapy mode, at step 995 the apparatus 1 determines if supplemental gas is being provided as part of the flow of gases. If supplemental gas is being provided then the apparatus continues to operate in the non-therapy mode, if supplemental gas is not being provided the apparatus proceeds to, at step 993, determine the output of the oxygen concentration sensor. At step 996, the apparatus 1 determines the oxygen concentration sensor calibration parameter based on the output of oxygen concentration sensor.

The oxygen concentration sensor parameter may additionally be determined based on the estimated oxygen ambient concentration which may, for example, be measured by another sensor, estimated, or input by a user via the user interface.

In some configurations, (for instance, when supplemental gases are provided as part of the flow of gases and/or when no ambient air is provided as part of the flow of gases) the oxygen concentration sensor calibration parameter may be determined based on the output of the oxygen concentration sensor and/or a predetermined oxygen concentration. The predetermined oxygen concentration may be for example 100%. In some configurations, the predetermined oxygen concentration may be entered by a user.

In some configurations, the apparatus may be configured such that no ambient air may be provided as part of the flow of gases. Configuring the apparatus 1, such that no ambient air may be provided as part of the flow of gases may be by controlling the at least one valve to provide only supplemental gas, and/or by prompting the user to take an action to prevent ambient air from being provided.

In some configurations, the user may be prompted to connect a supplementary source to the apparatus and indicate an oxygen concentration of the supplementary source.

After determination of the oxygen concentration sensor calibration parameter, the apparatus may be configured to run the flow generator at a predetermined flow rate, and/or at a predetermined motor speed. In some configurations, the apparatus may be configured to run the flow generator at a predetermined flow rate, and/or a predetermined motor speed after the apparatus has determined the oxygen concentration sensor calibration parameter. This may help to clear any oxygen from the system before the system enters a therapy mode.

The at least one sensor may be a flow rate sensor. The flow rate sensor may be configured to measure a flow rate of the flow of gases.

During the non-therapy mode, the apparatus may be configured to stop the flow generator from generating the flow of gases and determine an output of the flow rate sensor: the output may be indicative of the flow rate of the gases.

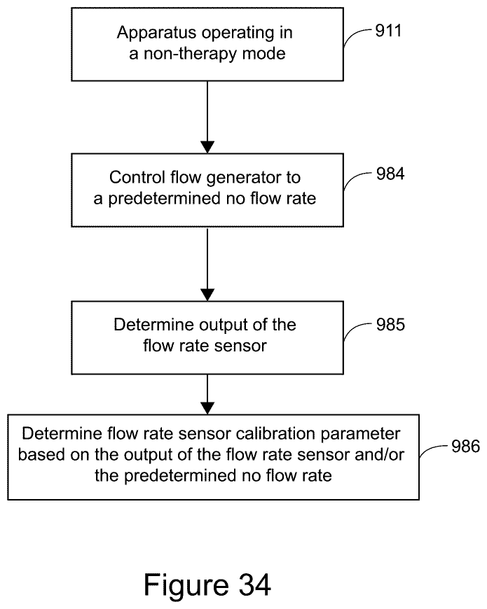

As shown in FIG. 34, the apparatus may be configured to, after controlling the flow generator to a no flow rate at step 984 determine a flow rate sensor calibration parameter at step 986 based on the output of the flow rate sensor (determined at step 985) and/or a predetermined no flow rate. The flow rate sensor calibration parameter may be applied to outputs of the flow rate sensor. The predetermined no flow rate may for example be 0 LPM.

During the non-therapy mode, the apparatus may be configured to, while the flow generator is generating the flow of gases, determine an output of the flow rate sensor. The output may be indicative of the flow rate of the gases. The apparatus may be configured to determine a flow rate sensor calibration parameter based on the output of the flow rate sensor and/or a predetermined flow rate. The parameter may be applied to outputs of the flow rate sensor. The predetermined flow rate may be above 0 LPM, or about 10 LPM, or about 20 LPM, or about 30 LPM, or about 40 LPM, or about 50 LPM, or about 60 LPM, or about 70 LPM.

The predetermined flow rate may be based on a motor speed (for example, from a motor speed sensor as described above). The relationship between the motor speed and flow rate may be based on a formula and/or look up table, such that a motor speed corresponds to an associated flow rate.

The apparatus may be configured to determine a flow rate sensor calibration parameter based on the output of the flow rate sensor and/or the motor speed. For example, as described above the motor speed would be expected to result in a known output of the flow rate sensor. For example, it may be known that at a particular motor speed (and optionally with no patient connected), a particular flow should be expected. If the flow rate sensor measures outside of predetermined tolerance, the flow rate sensor calibration parameter can be updated.

The allowable tolerance for the flow rate sensor may be about +/−0.1 LPM to about +/−3 LPM, or about +/−0.5 LPM to about +/−1 LPM.

The allowable tolerance with respect to the no flow rate may be the difference between the no flow rate and the output of the flow rate sensor when no flow is being provided.

The allowable tolerance with respect to the predetermined flow rate may be the difference between the predetermined flow rate and the output of the flow rate sensor at the predetermined flow rate.

It may be beneficial to determine a flow rate sensor calibration parameter during a non-therapy mode because non-therapeutic flow rates can be provided (for example, those not suitable for a patient or not as prescribed, for example, which could be lower or higher than typical therapeutic flow rates.

Further, during a non-therapy mode, the patient will not be connected, so the characteristics of the system will be consistent (for example, the flow conductance between the flow generator and the end of a conduit or disinfection conduit). Therefore, the relationship between a flow rate and motor speed, and/or between a measured flow rate and motor speed may be more reliable.

Further, in certain non-therapy modes such as disinfection mode, where the patient interface is disconnected and a specific tube with a known characteristics (such as flow conductance) is connected in a loop with the apparatus inlet/outlet, the comparison is even more reliable. Further, there may be no need to account for different interface types.

The apparatus may be configured to determine an output of the humidity sensor. The output may be indicative of a humidity of the flow of gases. In some configurations, the apparatus may be configured to determine the output of the humidity sensor (indicative of the humidity of the flow of gases) when supplemental gases are provided as the flow of gases. In some configurations, the apparatus may be configured to determine the output of the humidity sensor (indicative of the humidity of the flow of gases) when there is no ambient air in the flow of gasses.

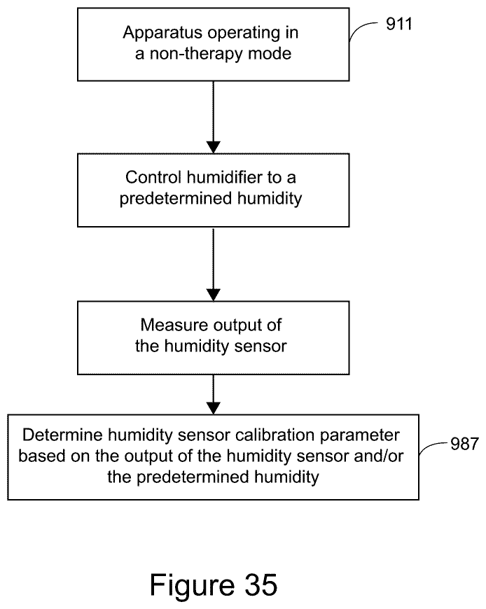

As shown in FIG. 35, in some configurations, the apparatus is configured to determine a humidity sensor calibration parameter at step 987 based on the output of the humidity sensor and/or a predetermined humidity. The predetermined humidity may be for example 0% relative humidity, or no absolute humidity. In some configurations, the controller may be configured to determine a humidity sensor calibration parameter based on an output of another humidity sensor.

The humidity sensor and/or the other humidity sensor may comprise an ambient humidity sensor or a gases flow humidity sensor.

In some configurations, the at least one sensor comprises a first humidity sensor and a second humidity sensor. The apparatus 1 may be configured to update the sensor calibration parameters relating to a first humidity sensor based on an output of a second humidity sensor. The sensor calibration parameters may be based on a comparison between an output of the first humidity sensor based on the output of a second humidity sensor.

The first humidity sensor may be an ambient humidity sensor and the second humidity sensor is a humidity sensor in located a flow path of the apparatus.

In the non-therapy mode, the controller may control the flow generator such that no flow of gases is provided from the flow generator so that the ambient humidity is the same as the pressure of the gases in the flow path (i.e., the flow path of the gases is the same humidity as ambient).

In some configurations, the first humidity sensor may be located in the same location as the second humidity sensor (for example, as ambient sensors, or located in the flow path of gases).

The allowable tolerance for the humidity sensor may be about +/−0% to about +/−5%, or about +/−0% to about +/−2%, or about +/−2%. The percentage may be a percentage of water vapour in the environment being sampled.

The allowable tolerance may be the difference between the output of the first humidity sensor and the output of the second humidity sensor.

The apparatus may be configured such that when it is operating in the at least one non-therapy mode, no therapy is provided to the user. The at least one non-therapy mode may comprise at least one of the following:
a drying mode configured to dry the conduit, and/or
a warm up mode, and/or
a standby mode.

The apparatus may be configured such that when it is operating in the at least one therapy mode, therapy is provided to the user. The at least one therapy mode may comprise at least one of the following:
a Continuous Positive Airway Pressure (CPAP) mode, and/or
a Bubble Continuous Positive Airway Pressure (BCPAP) mode, and/or
a Nasal High Flow (NHF) mode, and/or
a Bilevel (e.g., NIV) mode.

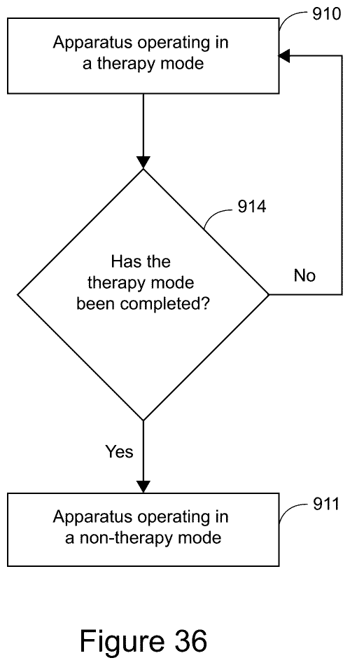

As shown in FIG. 36, in some configurations the apparatus is configured to automatically operate in the at least one non-therapy mode 911 after completion of the at least one therapy mode 914.

The apparatus may be configured to update parameters of the apparatus at the end of non therapy mode, and/or at the beginning of non-therapy mode.

1.-79. (canceled)

80. A breathing assistance apparatus comprising:
a flow generator configured to generate a flow of gases,
a humidifier configured to be pneumatically connected to the flow generator and to humidify the flow of gases,
wherein the apparatus is configured to be connected to a conduit that conveys the flow of gases,
wherein the apparatus is configured to operate in at least one therapy mode and at least one non-therapy mode, wherein when operating in the at least one therapy mode therapy is being provided to a user, and when operating in the at least one non-therapy mode, no therapy is being provided to the user,
wherein the apparatus is configured to collect and store data, the data comprising therapy data collected during operation in the at least one therapy mode or apparatus data,
wherein after a predetermined time operating in the at least one non-therapy mode, the apparatus is configured to:
transmit the collected and stored data to a device,
receive a software package,
receive therapy parameters, or
update parameters of the apparatus.

81. The breathing assistance apparatus of claim 80, wherein the at least one therapy mode comprises:
a continuous positive airway pressure (CPAP) mode,
a bubble continuous positive airway pressure (BCPAP) mode,
a nasal high flow (NHF) mode, or
a bilevel mode.

82. The breathing assistance apparatus of claim 80, wherein the apparatus is configured to automatically operate in the at least one non-therapy mode after completion of the at least one therapy mode.

83. The breathing assistance apparatus of claim 80, wherein the apparatus is configured to enter the at least one non-therapy mode after receiving an end of therapy command, wherein the end of therapy command is generated:
via an input from a user interface,
by detection that a patient interface has been removed from the user, or
by detection that the patient interface has been removed from the user for a predetermined amount of time.

84. The breathing assistance apparatus of claim 80, wherein when the apparatus is operating in the at least one non-therapy mode, the flow generator is activated and is generating the flow of gases and is providing the flow of gases at:
a flow rate less than a therapy flow rate of the flow of gases provided during a therapy mode, or
a predetermined flow rate.

85. The breathing assistance apparatus of claim 80, wherein the at least one non-therapy mode comprises a drying mode configured to dry the conduit and to evaporate remaining condensate in one or more of the apparatus, patient breathing conduit or patient interface, and wherein when the apparatus is operating in the drying mode at least one of the following occurs:
a heater of the conduit is controlled while the flow generator provides gases at a predetermined flow rate,
a heater of the humidifier is controlled to a predetermined value,
a heater plate is deactivated during the drying mode, or
the heater of the conduit is controlled to a predetermined temperature at an end of the conduit or controlled to a predetermined duty cycle, or to a predetermined voltage, or to a predetermined current or to a predetermined power.

86. The breathing assistance apparatus of claim 85, wherein the predetermined temperature is greater than 45 degrees Celsius.

87. The breathing assistance apparatus of claim 85, wherein the drying mode is configured to be operated for about 20 minutes to about 120 minutes, or about 90 minutes.

88. The breathing assistance apparatus of claim 85, wherein the drying mode comprises controlling the flow generator to provide a predetermined flow generator output, wherein the flow generator output is a motor speed of about 1000 RPM to about 3000 RPM or less than about 2000 RPM, and/or wherein the drying mode further comprises controlling the flow generator to provide a predetermined flow rate, wherein the predetermined flow rate is about 5 litres/minute to about 20 litres/minute.

89. The breathing assistance apparatus of claim 80, wherein the non-therapy mode is a warm up mode, wherein the apparatus operates in the warm up mode when the apparatus is powered on.

90. The breathing assistance apparatus of claim 89, wherein the warm up mode comprises controlling a heater of the conduit to control a temperature at an end of the conduit to a desired temperature, wherein the desired temperature at the end of the conduit is based on one or more therapy parameters of the apparatus and/or is a predetermined temperature, wherein the one or more therapy parameters are:
a therapy chamber outlet temperature,
a therapy dew point temperature at the chamber outlet,
a therapy dew point temperature at the end of the conduit,
a therapy humidity at the chamber outlet,
a therapy humidity at the end of the conduit, or
a therapy temperature at the end of the conduit.

91. The breathing assistance apparatus of claim 89, wherein the warm up mode comprises one or more of: deactivating the flow generator, controlling a heater of the humidifier to a predetermined temperature, controlling a heater of the humidifier to a predetermined duty cycle, controlling a heater of the humidifier to a predetermined voltage, controlling a heater of the humidifier to a predetermined current, controlling a heater of the humidifier to a predetermined power.

92. The breathing assistance apparatus of claim 80, wherein the non-therapy mode is a disinfection mode.

93. The breathing assistance apparatus of claim 92, wherein in the disinfection mode, the apparatus is configured to be connected to a disinfection conduit.

94. The breathing assistance apparatus of claim 93, wherein in the disinfection mode a heater of the disinfection conduit is controlled so that the flow of gases in the disinfection conduit reaches a predetermined temperature, wherein the predetermined temperature is about 50 degrees Celsius to about 100 degrees Celsius, or about 60 degrees Celsius to about 90 degrees Celsius.

95. The breathing assistance apparatus of claim 93, wherein the disinfection conduit comprises a temperature sensor.

96. The breathing assistance apparatus of claim 92, wherein the disinfection mode comprises controlling the flow generator to provide a predetermined flow rate, wherein the predetermined flow rate is about 10 litres/minute to about 20 litres/minute.

97. The breathing assistance apparatus of any one of claim 80, wherein the therapy data comprises data relating to the user or therapy provided to the user, wherein the therapy data comprises one or more of:
an oxygen saturation (SpO2) of the user,
a respiratory rate of the user,
a humidity (dew point) of the gases provided to the user,
a flow rate of the gases provided to the user,
a tidal volume of the user,
a minute ventilation of the user,
usage data of the apparatus,
answers to one of more questions provided to a user,
a therapy report, or
a results of a health enquiry.

98. The breathing assistance apparatus of claim 80, wherein the apparatus comprises one or more sensors configured to determine the therapy data, wherein when the apparatus is operating in the at least one non-therapy mode the user is presented with one or more questions, and provides answers to these questions via at least one user interface, and wherein the questions or answers to the questions form part of the therapy data, wherein the apparatus is configured to receive a sensor output from the one or more sensors, wherein the therapy data is based on the sensor output from one or more sensors.

99. The breathing assistance apparatus of claim 80, wherein the therapy data comprises data from at least one historic therapy mode or from at least one therapy mode which has not previously been transmitted to the device.

100. The breathing assistance apparatus of claim 80, wherein the data is transmitted to the device even if the apparatus has not operated in at least one therapy mode.

101. The breathing assistance apparatus of claim 80, wherein the data is transmitted to the device after a predetermined period of time has elapsed since a last transmission of data to the device, wherein the device is one or more of:
a server
a local device, or
a remote device.

102. The breathing assistance apparatus of claim 80, wherein the user is presented with a visual indicator and/or an audio indicator while the data is being transmitted to the device, or wherein the user is presented with a visual indicator and/or an audio indicator if the user attempts to power off the apparatus when the data is being transmitted to the device.

103. The breathing assistance apparatus of claim 80, wherein after receiving the software package, the apparatus applies the software package to the apparatus, wherein after receiving the therapy parameters, the apparatus updates the therapy parameters of the apparatus.

104. The breathing assistance apparatus of claim 80, wherein the apparatus only receives the software package or the therapy parameters if the software package or therapy parameters are an update to a current software package or a current therapy parameter.
